(19) United States
(12) Patent Application Publication
(10) Pub. No.: US 2024/0082334 A1
(43) Pub. Date: Mar. 14, 2024

(54) STABILIZED STEVIOL GLYCOSIDE COMPOSITIONS AND USES THEREOF

(71) Applicant: CARGILL, INCORPORATED, Wayzata, MN (US)

(72) Inventors: Dan S. GASPARD, Victoria, MN (US); Adam T. ZARTH, St. Louis Park, MN (US)

(73) Assignee: CARGILL, INCORPORATED, Wayzata, MN (US)

(21) Appl. No.: 18/335,968

(22) Filed: Jun. 15, 2023

Related U.S. Application Data
(63) Continuation of application No. 16/753,731, filed on Apr. 3, 2020, now abandoned.
(60) Provisional application No. 62/569,279, filed on Oct. 6, 2017.
(60) Provisional application No. 62/676,722, filed on May 25, 2018.

(51) Int. Cl. A61K 36/28; A23F 3/34; A23L 2/56; A23L 2/60; A23L 2/68; A23L 5/40; A23L 27/00; A23L 27/30; A61K 31/192; A61K 31/235; B01D 15/36; C07H 15/256; C07K 1/16; C07K 1/18

(52) U.S. Cl. CPC A61K 36/28; A23F 3/34; A23L 2/56; A23L 2/60; A23L 2/68; A23L 5/40; A23L 27/30; A23L 27/36; A23L 27/39; A23L 27/88; A61K 31/192; A61K 31/235; B01D 15/361; C07H 15/256; C07K 1/16; C07K 1/18; A23V 2002/00; A23V 2200/15; A23V 2250/258; A61K 2236/31; A61K 2236/39

(57) ABSTRACT

It has been surprisingly found that the presence of steviol glycoside stabilizing compounds, such as the solubility enhancers described herein, significantly increases the stability of steviol glycosides under most conditions, including highly acidic conditions (e.g., at a pH less than 2, such as the conditions to which SGs might be exposed to, in use, in a throw syrup) and/or at elevated temperatures (e.g., at temperatures exceeding 25° C., such as at 40° C.).

### CROSS-REFERENCE TO RELATED APPLICATIONS

This application is a Continuation of U.S. application Ser. No. 16/753,731, filed Apr. 3, 2020, which is a national phase application of PCT Application No. PCT/US2018/054696, filed Oct. 5, 2018, which claims the benefit of U.S. Provisional Application No. 62/569,279, filed Oct. 6, 2017, and U.S. Provisional Application No. 62/676,722, filed May 25, 2018, each of which is incorporated by reference in its entirety.

### BACKGROUND

Steviol glycosides (SGs) are currently being investigated as sweetening agents for use in foods, beverages, pharmaceuticals, and oral hygiene/cosmetic products, including in beverages such as carbonated soft drinks. The recent discovery of chlorogenic acids and cynarin isomers as solubility enhancers (SEs) for SGs is opening product lines that were previously unattainable, such as concentrated throw syrups and fountain drinks. While steviol glycosides can provide a non-caloric option for sweetening such products, there can be challenges to preparing such products with steviol glycosides. Though they can be stable at neutral pH, in some cases, steviol glycoside compositions can have limited chemical stability, especially at higher concentrations, over longer storage times at low pH and/or elevated temperatures. For example, beverage concentrates such as fountain syrups or throw syrups inherently have a low pH and can require storage over time, sometimes at elevated temperatures.

### SUMMARY

The disclosure provides, among other things, the use of steviol glycoside stabilizing compounds to enhance the chemical stability of SGs, such that the SGs can be used to prepare compositions with increased chemical stability, especially at higher SG concentrations and over longer storage times. The steviol glycoside stabilizing compounds can also enhance the chemical stability of SGs under acidic conditions and/or at elevated temperatures. This disclosure also describes the enhanced chemical stability of the steviol glycoside stabilizing compounds themselves, which are less subject to acidic hydrolysis and/or oxidation in the presence of SGs. This effect appears to be concentration dependent, with higher concentrations yielding greater stability. This finding opens up the possibility of using SGs in water enhancers, coffee syrups, and liquid stevia products where the SG concentration is higher, the desired shelf-life is longer, and/or the pH can be acidic.

While not wishing to be bound by any theory, it is hypothesized that the mechanism by which the steviol glycoside stabilizing compounds increase the chemical stability of SGs in solution has to do, in part, by reducing or altering the interactions between water molecules and SG molecules. It is also hypothesized that the interactions with strong acids are also reduced, effectively shielding the SGs from acidic hydrolysis.

### DESCRIPTION OF THE DRAWINGS

The drawings illustrate generally, by way of example, but not by way of limitation, various embodiments discussed herein.

FIG. 1 is a flow diagram of an example of a method for making a composition comprising steviol glycoside stabilizing compounds, such as caffeic acid, monocaffeoylquinic acids, and dicaffeoylquinic acids, and salts thereof.

FIG. 2 is a flow diagram of another example of a method for making a composition comprising steviol glycoside stabilizing compounds, such as caffeic acid, monocaffeoylquinic acids, and dicaffeoylquinic acids, and salts thereof.

FIG. 3 is a flow diagram of another example of a method for making a composition comprising steviol glycoside stabilizing compounds, such as caffeic acid, monocaffeoylquinic acids, and dicaffeoylquinic acids, and salts thereof.

FIG. 4 is a flow diagram of another example of a method for making a composition comprising steviol glycoside stabilizing compounds, such as caffeic acid, monocaffeoylquinic acids, and dicaffeoylquinic acids, and salts thereof.

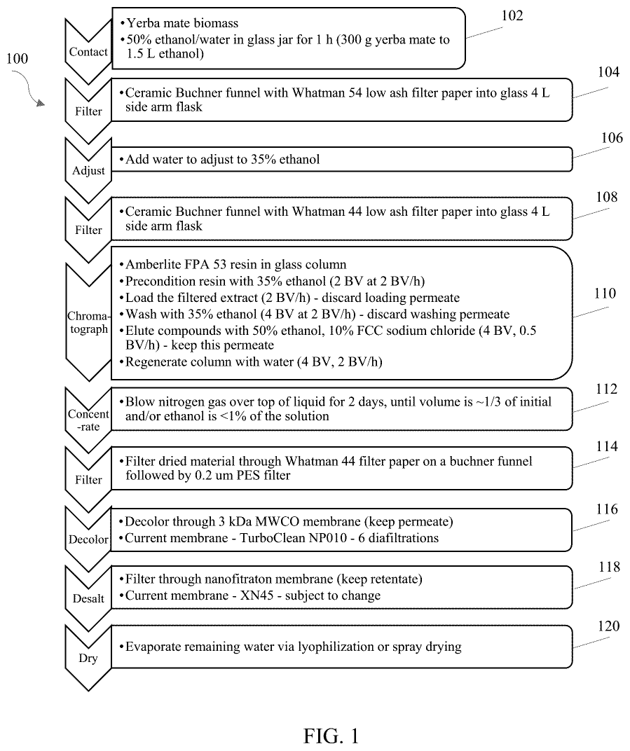

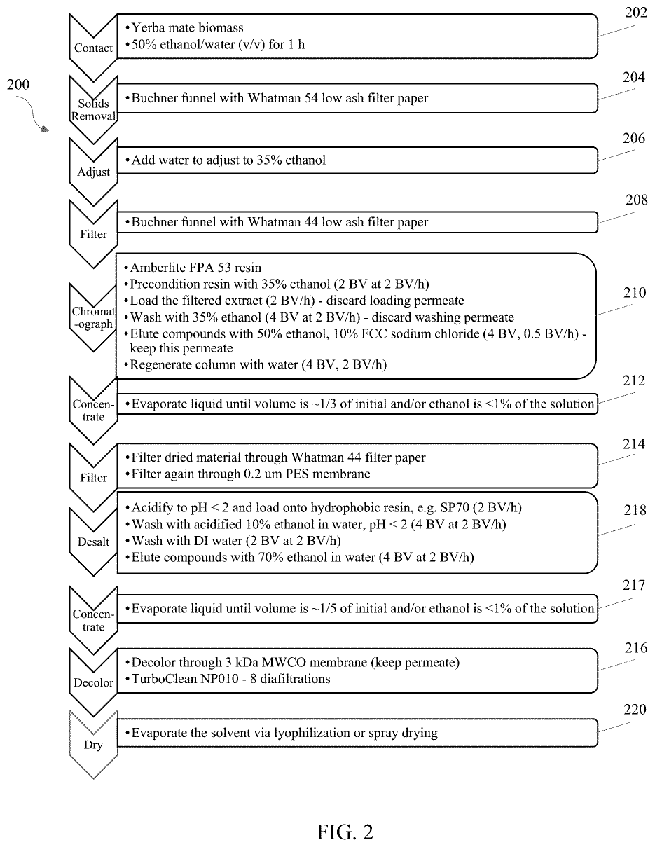

Repeated use of reference characters in the specification and drawings is intended to represent the same or analogous features or elements of the disclosure, even when the numbers increase by 100 from figure-to-figure (e.g., drying operation 120 in FIG. 1 is analogous to or the same as drying operations 220, 320, and 420 in FIGS. 2-4, respectively). It should be understood that numerous other modifications and examples can be devised by those skilled in the art, which fall within the scope and spirit of the principles of the disclosure.

### DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The disclosure relates generally to compositions comprising a steviol glycoside (SG) and a steviol glycoside stabilizing compound in an amount effective to reduce degradation of the SG. The compositions comprising the SG and the steviol glycoside stabilizing compound can be formulated in any suitable way, including as an aqueous composition.

It has been surprisingly found that the presence of steviol glycoside stabilizing compounds significantly increases the stability of SGs under most conditions, including at higher concentrations of SG and over longer storage times. Additionally, the presence of steviol glycoside stabilizing compounds significantly increases the stability of SGs under acidic conditions and/or at elevated temperatures. For example, in the presence of the steviol glycoside stabilizing compounds described herein, one can not only access SG concentrations of up to 35 wt. % (e.g., from about 1 wt. % to about 35 wt. %, with 5 wt. % being a suitable concentration for a liquid stevia application), but the SGs will be chemically stable at acidic pH over a period of greater than 72 days, or even for a period of one year or longer. In sum, the SG/steviol glycoside stabilizing compound compositions are chemically stable for weeks, months or even years, even at acidic pHs and/or elevated temperatures.

The term “chemical stability” refers to a reduced chemical degradation of SGs, including hydrolysis and isomerization of the double bond on the steviol core. For example, a chemically stable Rebaudioside M (Reb M) resists degradation to hydrolysis products such as Rebaudioside A (Reb A), Rebaudioside B (Reb B), iso-Rebaudioside M (iso-Reb M), iso-Rebaudioside A (iso-Reb A), and iso-Rebaudioside B (iso-Reb B). Chemical stability can be measured by known methods such as by UHPLC analysis. For example, as described in the Examples herein, a composition comprising Reb M can be injected directly for analysis by UHPLC-UV. The chromatographic analysis can be performed on a C18-based reversed-phase chromatography column at elevated temperature under gradient conditions, utilizing trifluoroacetic acid in water and acetonitrile. SGs can be detected utilizing a UV detector set to 210 nm. A linear calibration curve can be applied using Reb A standard as a reference solution.

In some aspects, the amount of steviol glycoside stabilizing compound effective to reduce degradation of SG is an amount to chemically stabilize the SG over higher concentrations of SG and/or over longer storage times. For example, the amount of SG and/or steviol glycoside stabilizing compound can be at least 1 wt. %, at least 3 wt. %, at least 5 wt. %, at least 10 wt. %, at least 15 wt. %, at least 20 wt. %, at least 30 wt. %; about 1 wt. % to about 35 wt. %, about 5 wt. % to about 15 wt. %, about 1 wt. % to about 5 wt. % or about 5 wt. % to about 20 wt. %, about. And within these stated ranges, the SG and the steviol glycoside stabilizing compound can be chemically stable for days, weeks, months or years.

In some aspects, the amount of steviol glycoside stabilizing compound effective to reduce degradation of SG can be determined by an accelerated chemical stability assay. For example, the “amount of steviol glycoside stabilizing compound effective to reduce degradation of the steviol glycoside” is an amount such that at least about 10% (e.g., at least about 20%, at least about 30%, at least about 40%, at least about 50%, at least about 60%, at least about 70%, at least about 80%, at least about 90%, at least about 95%; or from about 10% to about 100%, about 20% to about 80%, about 30% to about 95%, about 40% to about 80%, about 60% to about 90% or about 70% to about 99% or more) relative to an initial steviol glycoside concentration remains when the stabilized steviol glycoside composition is subjected to storage for 7 days at 40° C. in a 5% phosphoric acid solution. In some cases, there is statistically less degradation of the steviol glycosides than when steviol glycoside stabilizing compound is absent.

The compositions comprising a SG and a steviol glycoside stabilizing compound can have a pH of less than about 5 (e.g., less than about 4, less than about 3, less than about 2.5, less than about 2, less than about 1.7, less than about 1.5, less than about 1, much less than about 1; about 0.1 to about 4, about 1 to about 4, about 0.5 to about 2 or about 1 to about 3).

The compositions comprising a SG and a steviol glycoside stabilizing compound are storable at room temperature, at below room temperature (e.g., 4° C.) or at above room temperature (e.g., at 40° C.), without any substantial degradation of the steviol glycoside.

The compositions comprising a SG and a steviol glycoside stabilizing compound can comprise any suitable steviol glycoside, such as stevioside, Reb A, Reb C, dulcoside A, Reb M, Reb B, Reb D, and Reb E and salts thereof (in cases where compounds can form salts, such as in the case of Reb B, steviobioside, and steviol-13-O-glucoside (13-SMG)). The steviol glycoside can be Reb M, Reb A or mixtures of one or more of the aforementioned steviol glycosides. In one aspect, the steviol glycoside comprises one or more steviol glycosides selected from the list consisting of Reb A, Reb B, Reb C, Reb D, Reb E, Reb F, Reb M, Reb N, Reb O, rubusoside, dulcoside A, Reb I, Reb Q, 1,2-stevioside, 1,3-stevioside, steviol-1,2-bioside, steviol-1,3-bioside, 13-SMG, steviol-19-O-glucoside (19-SMG), and steviol glycosides with 1, 2, 3, 4, 5, 6, 7, 8, 9, 10 or more sugar additions, such as glucose, rhamnose, or xylose as three examples.

The compositions comprising a SG and a steviol glycoside stabilizing compound can comprise any suitable steviol glycoside at any suitable concentration. For example, the compositions comprising a SG and a steviol glycoside stabilizing compound can comprise any suitable amount of steviol glycoside, such as at least about 0.03 wt. % (e.g., at least about 0.1 wt. %, at least about 0.5 wt. %, at least about 0.6 wt. %, at least about 1 wt. %, at least about 1.5 wt. %, at least about 2.5 wt. %, at least 3 wt. %, at least about 5 wt. %, at least about 6 wt. %, at least about 10 wt. %, at least about 15 wt. %, at least about 20 wt. %, at least about 25 wt. %, at least about 30 wt. %, at least about 35 wt. %, or up to about 40 wt. %; about 0.03 wt. % to about 5 wt. %, about 0.1 wt. % to about 10 wt. %, about 1 wt. % to about 4 wt. %, about 0.1 wt. % to about 5 wt. %, about or about 1 wt. % to about 5 wt. %) steviol glycoside.

The compositions comprising a SG and a steviol glycoside stabilizing compound can comprise any suitable steviol glycoside at any suitable concentration. The compositions comprising a SG and a steviol glycoside stabilizing compound can comprise concentrated products that are diluted by the consumer for consumer use. For example, beverage concentrates such as throw syrups are diluted six to seven times for use and flavored water enhancer liquids are diluted up to 100 times.

For example, for a beverage concentrate product comprising the compositions comprising a SG and a steviol glycoside stabilizing compound can comprise between about 1,800 ppm and about 10,000 ppm (e.g., about 2,000 ppm to about 5,000 ppm, about 2,000 ppm to about 8,000 ppm, about 3,000 ppm to about 5,000 ppm or about 2,500 ppm to about 7,500 ppm) steviol glycoside.

In another example, this time for a liquid water enhancer product, the compositions comprising a SG and a steviol glycoside stabilizing compound can comprise between about 1.5 wt. % and about 3.5 wt. %. (e.g., about 2 wt. % to about 3 wt. %, about 1.5 wt. % to about 2 wt. % or about 2.5 wt. % to about 3.5 wt. %) steviol glycoside.

In yet another example, this time for a liquid sweetener, the compositions comprising a SG and a steviol glycoside stabilizing compound can comprise between about 1.0 wt. % and about 10 wt. % (e.g., about 4 wt. % to about 10 wt. %, about 5 wt. % to about 10 wt. %, about 7 wt. % to about 9 wt. % or about 8 wt. % to about 10 wt. %) steviol glycoside.

In some aspects, the compositions comprising a SG and a steviol glycoside stabilizing compound can comprise any suitable additives including buffering agent, acidulants, such as citric acid, antimicrobial agents, such as benzoic acid and sorbic acid (and salts thereof), natural colors, natural flavors, artificial flavors, artificial colors, and artificial sweeteners.

Examples of steviol glycoside stabilizing compounds include:
caffeic acid, an ester of caffeic acid, an ester of caffeic acid and quinic acid, an ester of caffeic acid and quinic acid comprising a single caffeic acid moiety (e.g., chlorogenic, cryptochlorogenic, and neochlorogenic acid; structures of each are provided herein), an ester of caffeic acid and quinic acid comprising more than one caffeic acid moiety (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid; structures of each are provided herein);
ferulic acid, an ester of ferulic acid, an ester of ferulic acid and quinic acid, an ester of ferulic acid and quinic acid comprising a single ferulic acid moiety, an ester of ferulic acid and quinic acid comprising more than one ferulic acid moiety;
3-(3,4-dihydroxyphenyl)lactic acid, a 3-(3,4-dihydroxyphenyl)lactic acid derivative, an ester of 3-(3,4-dihydroxyphenyl)lactic acid, an ester of a 3-(3,4-dihydroxyphenyl)lactic acid derivative,
quinic acid, a quinic acid derivative, an ester of quinic acid, an ester of a quinic acid derivative;
p-coumaric acid, an ester of p-coumaric acid, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety;
sinapic acid, an ester of sinapic acid, an ester of sinapic acid and quinic acid, an ester of sinapic acid and quinic acid comprising a single sinapic acid moiety, an ester of sinapic acid and quinic acid comprising more than one sinapic acid moiety;
tartaric acid, a tartaric acid derivative, an ester of tartaric acid, an ester of a tartaric acid derivative, and
3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid.

Caffeic acid has the structure:

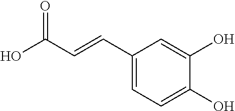 [SMILES: O=C(O)/C=C/c1ccc(O)c(O)c1]

Ferulic acid has the structure:

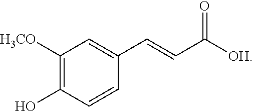 [SMILES: COc1cc(/C=C/C(=O)O)ccc1O]

p-Coumaric acid has the structure:

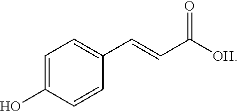 [SMILES: O=C(O)/C=C/c1ccc(O)cc1]

Sinapic acid has the structure:

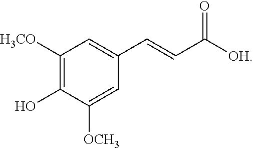 [SMILES: COc1cc(/C=C/C(=O)O)cc(OC)c1O]

Quinic acid has the structure:

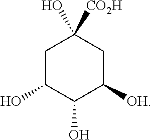 [SMILES: O=C(O)[C@]1(O)C[C@@H](O)[C@@H](O)[C@H](O)C1]

3-(3,4-dihydroxyphenyl)lactic acid has the structure:

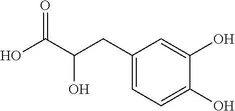 [SMILES: O=C(O)C(O)Cc1ccc(O)c(O)c1]

Tartaric acid has the structure:

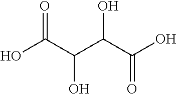

and can be in the D and L forms.

Examples of the esters of the various acids contemplated herein include the ester of caffeic acid and quinic acid, which includes monocaffeoylquinic acids (e.g., chlorogenic acid, neochlorogenic acid, and cryptochlorogenic acid), and dicaffeoylquinic acids (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid), and salts thereof:

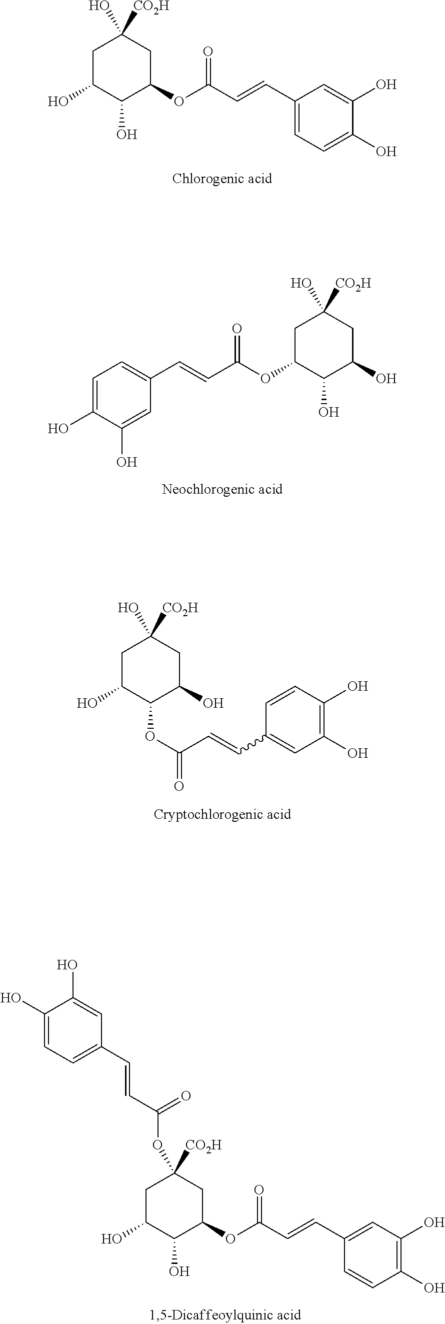 [SMILES: O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@@H]1C[C@@](O)(C(=O)O)C[C@@H](O)[C@@H]1O ; O=C(C=Cc1ccc(O)c(O)c1)O[C@H]1[C@H](O)C[C@](O)(C(=O)O)C[C@H]1O ; O=C(/C=C/c1ccc(O)c(O)c1)O[C@@H]1C[C@](OC(=O)/C=C/c2ccc(O)c(O)c2)(C(=O)O)C[C@@H](O)[C@H]1O]

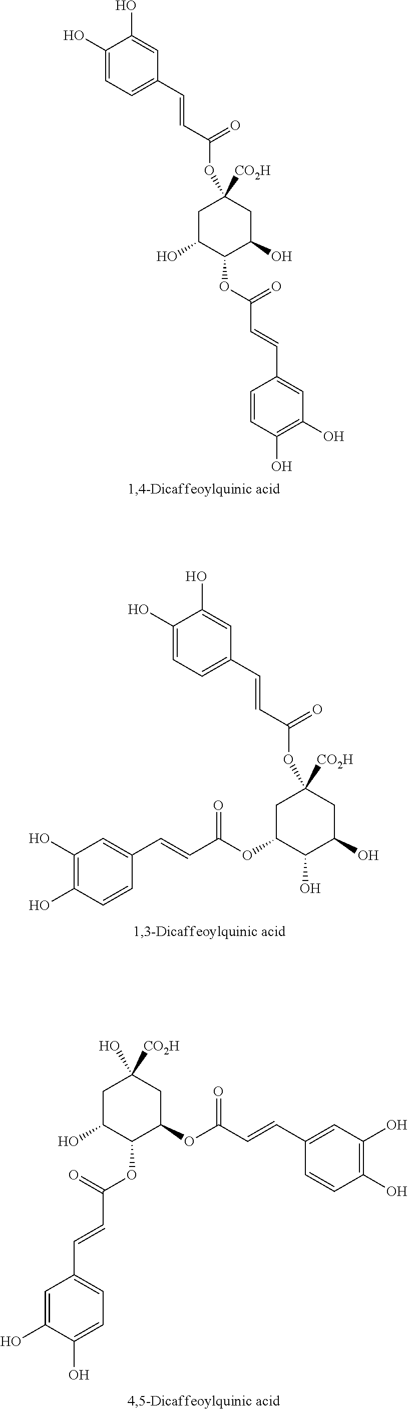

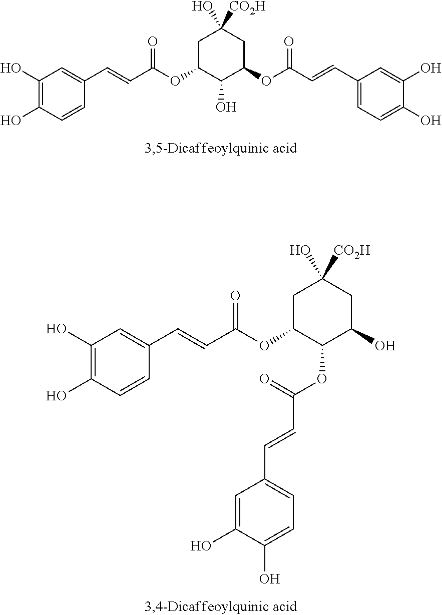 [SMILES: O=C(/C=C/c1ccc(O)c(O)c1)O[C@@H]1C[C@](O)(C(=O)O)C[C@@H](OC(=O)/C=C/c2ccc(O)c(O)c2)[C@H]1O ; O=C(/C=C/c1ccc(O)c(O)c1)O[C@H]1[C@H](O)C[C@](O)(C(=O)O)C[C@H]1OC(=O)/C=C/c1ccc(O)c(O)c1]

with 4,5-dicaffeoylquinic acid, 3,5-dicaffeoylquinic acid, and 3,4-dicaffeoylquinic acid being most prevalent in the compositions contemplated herein and most prevalent in abundant in stevia, yerba mate, globe artichoke, and green coffee.

Examples of the esters of the various acids contemplated herein include the ester of caffeic acid and tartaric acid, which includes cichoric acid having the structure:

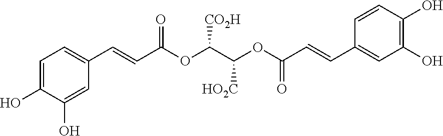 [SMILES: O=C(/C=C/c1ccc(O)c(O)c1)O[C@H](C(=O)O)[C@H](OC(=O)/C=C/c1ccc(O)c(O)c1)C(=O)O]

which has two caffeic acid molecules linked to a tartaric acid core and caftaric acid having the structure:

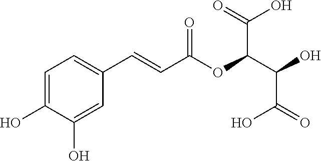 [SMILES: O=C(/C=C/c1ccc(O)c(O)c1)O[C@@H](C(=O)O)[C@@H](O)C(=O)O]

which has one caffeic acid molecule linked to a tartaric acid core.

Examples of the esters of the various acids contemplated herein include the ester of caffeic acid and 3-(3,4-dihydroxyphenyl)lactic acid including, for example, rosmarinic acid, which has the structure:

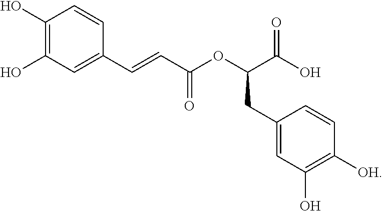 [SMILES: O=C(/C=C/c1ccc(O)c(O)c1)O[C@H](Cc1ccc(O)c(O)c1)C(=O)O]

Some or all of these steviol glycoside stabilizing compounds can be isolated from botanical sources, including but not limited to botanical sources such as eucommoia ulmoides, honeysuckle, nicotiana benthamiana, globe artichoke, cardoon, stevia Rebaudiana, monkfruit, coffee, coffee beans, green coffee beans, tea, white tea, yellow tea, green tea, oolong tea, black tea, red tea, post-fermented tea, bamboo, heather, sunflower, blueberries, cranberries, bilberries, grouseberries, whortleberry, lingonberry, cowberry, huckleberry, grapes, chicory, eastern purple coneflower, echinacea, Eastern pellitory-of-the-wall, Upright pellitory, Lichwort, Greater celandine, Tetterwort, Nipplewort, Swallowwort, Bloodroot, Common nettle, Stinging nettle, Potato, Potato leaves, Eggplant, Aubergine, Tomato, Cherry tomato, Bitter apple, Thorn apple, Sweet potato, apple, Peach, Nectarine, Cherry, Sour cherry, Wild cherry, Apricot, Almond, Plum, Prune, Holly, Yerba mate, Mate, Guayusa, Yaupon Holly, Kuding, Guarana, Cocoa, Cocoa bean, Cacao, Cacao bean, Kola nut, Kola tree, Cola nut, Cola tree, Ostrich fern, Oriental ostrich fern, Fiddlehead fern, Shuttlecock fern, Oriental ostrich fern, Asian royal fern, Royal fern, Bracken, Brake, Common bracken, Eagle fern, Eastern brakenfern, Clove, Cinnamon, Indian bay leaf, Nutmeg, Bay laurel, Bay leaf, Basil, Great basil, Saint-Joseph's-wort, Thyme, Sage, Garden sage, Common sage, Culinary sage, Rosemary, Oregano, Wild marjoram, Marjoram, Sweet marjoram, Knotted marjoram, Pot marjoram, Dill, Anise, Star anise, Fennel, Florence fennel, Tarragon, Estragon, Mugwort, Licorice, Liquorice, Soy, Soybean, Soyabean, Soya vean, Wheat, Common wheat, Rice, Canola, Broccoli, Cauliflower, Cabbage, Bok choy, Kale, Collard greens, Brussels sprouts, Kohlrabi, Winter's bark, Elderflower, Assa-Peixe, Greater burdock, Valerian, and Chamomile

The compositions comprising a SG and a steviol glycoside stabilizing compound can comprise any suitable amount of steviol glycoside stabilizing compound, such as at least about 0.03 wt. % (e.g., at least about 0.1 wt. %, at least about 0.5 wt. %, at least about 0.6 wt. %, at least about 1 wt. %, at least about 1.5 wt. %, at least about 2.5 wt. %, at least 3 wt. %, at least about 5 wt. %, at least about 6 wt. %, at least about 10 wt. %, at least about 15 wt. %, at least about 20 wt. %, at least about 25 wt. %, at least about 30 wt. %, at least about 35 wt. %, or up to about 40 wt. %; about 0.03 wt. % to about 5 wt. %, about 0.1 wt. % to about 10 wt. %, about 1 wt. % to about 4 wt. %, about 0.1 wt. % to about 5 wt. %, about or about 1 wt. % to about 5 wt. %) steviol glycoside stabilizing compound.

The compositions comprising a SG and a steviol glycoside stabilizing compound can comprise a 1:0.3 to 1:3 (e.g., 1:1 to 1:1.5; or 1:1 to 1:2) ratio by weight of steviol glycoside to steviol glycoside stabilizing compound.

Also contemplated herein are diastereomers and structural isomers of any of the aforementioned acids. And because the aforementioned acids can be considered weak acids, they can each exist in at least one of their conjugate acid form, conjugate base form (e.g., in their salt form), and mixed conjugate acid-conjugate base form, wherein a fraction (e.g., mole fraction) of the compounds exist in the conjugate acid form and another fraction exist in the conjugate base form. The fraction of conjugate acid form to conjugate base of each acid will depend on various factors, including the pKₐ of each compound and the pH of the composition. Examples of salts of any of the aforementioned acids include, but are not limited to, quaternary ammonium, sodium, potassium, lithium, magnesium, and calcium salts of the one or more steviol glycoside stabilizing compounds, and the like.

An example of a method for making a composition comprising one or more steviol glycoside stabilizing compounds, and salts thereof, comprises:
(a) contacting yerba mate biomass with an aqueous composition to obtain an initial extract;
(b) removing solids from the initial extract to obtain a second initial extract;
(c) adjusting the volume of the second initial extract with an aqueous composition to obtain an adjusted second initial extract;
(d) chromatographing the adjusted second initial extract on an ion exchange chromatography stationary phase;
(e) eluting the ion exchange chromatography stationary phase to obtain a first eluent comprising a solvent;
(f) removing the solvent to form a concentrate; and
(g) at least one of decoloring and desalting the concentrate to at least one of a filtrate and a retentate.

Another example of a method for making a composition comprising one or more steviol glycoside stabilizing compounds, and salts thereof, comprises:
(a) contacting yerba mate biomass with an aqueous composition to obtain an initial extract;
(b) removing solids from the initial extract to obtain a second initial extract;
(c) adjusting the volume of the second initial extract with an aqueous composition to obtain an adjusted second initial extract;
(d) chromatographing the adjusted initial extract on an ion exchange chromatography stationary phase;
(e) eluting the ion exchange stationary phase to obtain a first eluent comprising a solvent;
(f) removing the solvent to form a concentrate;
(g) at least one of decoloring and desalting the concentrate to obtain at least one of a filtrate and a retentate; and
(h) drying the at least one of a filtrate and a retentate to obtain the composition comprising one or more steviol glycoside stabilizing compounds, and salts thereof.

Step (a) of the methods described herein involve contacting yerba mate biomass with an aqueous composition to obtain an initial extract comprising one or more steviol glycoside stabilizing compounds, and salts thereof (e.g., quaternary ammonium, sodium, potassium, lithium, magnesium, and calcium salts).

The aqueous composition can comprise water and not contain any co-solvents, such as organic solvents. But the aqueous composition can comprise co-solvents, in addition to water. Suitable co-solvents include organic solvents, such as, (C₁-C₄)alkanols and mixtures of (C₁-C₄)alkanols. By “(C₁-C₄)alkanol” is meant an alcohol of the formula (C₁-C₄)alkyl-OH, wherein “alkyl” refers to straight chain and branched alkyl groups having from 1 to 4 carbon atoms such as methyl, ethyl, n-propyl, n-butyl, isopropyl, iso-butyl, sec-butyl, and t-butyl, such that the resulting (C₁-C₄)alkanol is methanol, ethanol, n-propanol, n-butanol, isopropanol, iso-butanol, sec-butanol, and t-butanol. The proportion of organic solvent, such as (C₁-C₄)alkanol or mixtures of (C₁-C₄)alkanols, can be any suitable proportion such that the aqueous composition can comprise up to about 30%, up to about 40%, up to about 50% or up to about 60%, up to about 70%, up to about 80%, up to about 90% or up to 100% by volume organic solvent the balance being water, except when the aqueous composition comprises 100% by volume organic solvent; or from about 30% to about 100%, about 50% to about 100%, about 60% to about 90%, about 30% to about 60%, about 40% to about 60%, about 30% to about 50%, about 40% to about 50%, or about 50% by volume organic solvent, the balance being water.

In some instances, the aqueous composition can be buffered with any suitable buffering system, including, but not limited to, a phosphate, citrate, ascorbate, lactate, acetate, and the like. Buffers can be in the range of 1-1000 mM of the anion. Alternatively, water acidified to pH 5-6 with hydrochloric acid, sulfuric acid, nitric acid or the like can be useful in the aqueous composition, with or without a co-solvent. Alternatively pure water made basic to pH 7-11 with hydroxide, such as with sodium or potassium hydroxide, can be useful in the aqueous composition, with or without a co-solvent. In still other instances, it may be suitable to add a suitable non-ionic solute that can help balance the osmotic potential of the aqueous composition.

As used herein, the term “yerba mate biomass” generally refers to any and all parts of the yerba mate plant, such as Ilex paraguariensis, including the yerba mate plant leaves, stalks, stems, tops, roots, and the like. The yerba mate biomass can be in any suitable form including in comminuted form resulting from, e.g., from chopping the yerba mate biomass prior to and/or during the contacting with the aqueous composition. For example, the yerba mate biomass can be comminuted in a suitable container and the aqueous composition can be added to the comminuted yerba mate biomass, thus “contacting” the yerba mate biomass. The comminuted yerba mate biomass can then be optionally further comminuted within the suitable container. Or the yerba mate biomass can be placed in a suitable container, to which the aqueous composition is added, thus “contacting” the yerba mate biomass, and the resulting composition can be comminuted.

The yerba mate biomass can be stirred, sonicated or otherwise agitated prior to and/or during the contacting to, among other things, maximize the extraction of, among other of the acids described herein, the one or more steviol glycoside stabilizing compounds, and salts thereof.

The initial extract can be carried through to step (c) as-is or bulk solids and or plant solids present, such as comminuted yerba mate plant leaves, stalks, tops, roots, and the like, can be removed in step (b) of the methods described herein. When step (b) is carried out, one obtains a second initial extract.

Bulk solids can be removed by any suitable method, including centrifugation, skimming, or filtration. For example, the initial extract can be filtered using any suitable filtration method, including gravity filtration or vacuum filtration through any suitable filter, so long as the filter does not substantially retain the one or more steviol glycoside stabilizing compounds, and salts thereof, including a paper filter (e.g., low ash filter paper, such as Whatman 44 or 54 low ash filter paper), a nylon filter, polyethersulfone filter, a glass fiber filter, a pad of diatomaceous earth, and the like.

Step (c) of the methods described herein involves adjusting the volume of the initial extract or second initial extract with a first aqueous composition or a second aqueous composition, respectively, to obtain an adjusted initial extract or adjusted second initial extract. The first and second aqueous compositions can be different or the same. The adjusted initial extract or adjusted second initial extract can be filtered at this point or can be carried through to step (d) as-is. The initial extract or the second initial extract can be filtered using any suitable filtration method, including gravity filtration or vacuum filtration through any suitable filter, so long as the filter does not substantially retain the one or more steviol glycoside stabilizing compounds, and salts thereof, including a paper filter (e.g., low ash filter paper, such as Whatman 44 or 54 low ash filter paper), a nylon filter, polyethersulfone filter, a glass fiber filter, a pad of diatomaceous earth, and the like.

The volume of the initial extract or second initial extract can be adjusted with a sufficient amount of an aqueous composition (e.g., water) to obtain an adjusted initial extract or adjusted second initial extract to, among other things, increase the binding of the one or more steviol glycoside stabilizing compounds, and salts thereof, to the ion exchange chromatography column used in step (d) of the methods described herein, relative to an unadjusted initial extract or an unadjusted second initial extract.

The volume of the initial extract or second initial extract can be adjusted to, among other things, adjust the amount of organic solvent, when present, in the initial extract or second initial extract. The volume of the initial extract or second initial extract can be adjusted such that the adjusted initial extract or adjusted second initial extract comprises less than about 60%, less than about 50%, less than about 40%, less than about 30%, less than about 20%, less than about 10%, less than about 5%, less than about 1% or even about 0% by volume organic solvent, the balance being water; or from about 0% to about 40%, about 0% to about 30%, about 10% to about 40%, about 10% to about 30%, about 20% to about 40%, about 30% to about 40%, or about 35% by volume organic solvent, the balance being water.

Step (d) of the methods described herein involves chromatographing the adjusted initial extract or the second initial extract on an ion exchange stationary phase (e.g., a weak anion exchange stationary phase). The chromatographing can be performed in any suitable fashion, including in batch mode or using a column. The chromatographing can be performed with an aqueous composition (e.g., an aqueous composition comprising a (C₁-C₄)alkanol) as eluent (e.g., an aqueous composition comprising from about 0% to about 40%, about 0% to about 30%, about 10% to about 40%, about 10% to about 30%, about 20% to about 40%, about 30% to about 40%, or about 35% by volume (C₁-C₄)alkanol, the balance being water), leaving one or more steviol glycoside stabilizing compounds, and salts thereof, adsorbed on the weak ion exchange chromatography column, while eluting other compounds including caffeine, rutin (also known as rutoside, quercetin-3-O-rutinoside, and sophorin)

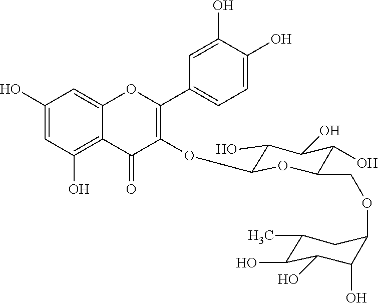 [SMILES: CC1CC(OCC2OC(Oc3c(-c4ccc(O)c(O)c4)oc4cc(O)cc(O)c4c3=O)C(O)C(O)C2O)C(O)C(O)C1O]

and isomers thereof. Step (d) of the methods described herein can decrease the concentration of at least one of caffeine, rutin, and rutin isomers to a concentration of less than 1%, less than 0.5%, less than 0.1%, less than 0.05%, less than 0.01% or less than 0.001% by mass. The instant disclosure therefore contemplates yerba mate extracts comprising less than 0.1% of at least one of caffeine, rutin, and rutin isomers by mass. The instant disclosure also contemplates yerba mate extracts comprising less than 0.5% by mass of each one of caffeine, rutin, and rutin isomers and a less than about 1% by mass of caffeine, rutin, and rutin isomers combined. The instant disclosure also contemplates yerba mate extracts that are effectively free of at least one of caffeine, rutin, and rutin isomers (e.g., free of caffeine, free of rutin, free of rutin isomers, and/or free of caffeine, rutin, and rutin isomers).

The ion exchange stationary phase is non-limiting and can be any suitable ion exchange chromatography stationary phase. Examples of suitable ion exchange chromatography stationary phases include ANX-SEPHAROSE® fast flow resin, DEAE SEPHAROSE®, DEAE SEPHADEX® A25 resin, Relite RAM2, Relite MG1P, AMBERLITE® (FPA 53; FPA 55; CG-50 Type I; IRC-50; IRC-50S; and IRP-64), DIAION WA10, and DOWEX® CCR-3.

The ion exchange chromatography stationary phase can optionally be pre-conditioned with an aqueous composition (e.g., an aqueous composition comprising a (C₁-C₄)alkanol), such as an aqueous composition comprising from about 0% to about 40%, about 0% to about 30%, about 10% to about 40%, about 10% to about 30%, about 20% to about 40%, about 30% to about 40%, or about 35% by volume (C₁-C₄)alkanol, the balance being water, prior to the chromatographing of the adjusted initial extract or adjusted second initial extract. For example, the weak ion exchange chromatography column can be pre-conditioned with about 2 or more bed volumes (BV) at a flow rate of about 2 BV/h.

The pH of the weak ion exchange chromatography column can optionally be adjusted prior to the chromatographing of the adjusted initial extract or adjusted second initial extract. For example, the pH of the weak ion exchange chromatography column can be adjusted prior to the chromatographing with any suitable acid (e.g., hydrochloric acid) such that the pH of the weak ion exchange chromatography column (e.g., the pH of the resin/stationary phase) is a pH of less than about 10, about 9 or less, about 8 or less, about 7 or less, about 6 or less, about 5 or less, about 4 or less, about 3 or less; or a pH of about 2 to about 10, about 3 to about 8, about 5 to about 9, about 2 to about 6; about 3 to about 4; or about 3 to about 6. The pH of the weak ion exchange chromatography column can be adjusted before or after the column is optionally pre-conditioned with the aqueous composition comprising a (C₁-C₄) prior to the chromatographing of the adjusted initial extract or adjusted second initial extract.

After pre-conditioning and/or adjusting of the pH of the weak ion exchange chromatography column, the adjusted initial extract or adjusted second initial extract can be loaded onto the column at any suitable rate, such as at a rate of above 2 BV/h (bed volumes per hour). After loading the adjusted initial extract or adjusted second initial extract, the column can be washed with any suitable volume of an aqueous composition comprising a (C₁-C₄)alkanol (e.g., at least about 2 BV, at least about 3 BV or at least about 4 BV of an aqueous composition comprising from about 10% to about 40%, about 10% to about 30%, about 20% to about 40%, about 30% to about 40%, or about 35% by volume (C₁-C₄)alkanol, the balance being water) at any suitable rate, such as at a rate of about 2 BV/h. The volume of aqueous composition comprising a (C₁-C₄)alkanol can be discarded, as it will contain, among other things, caffeine, rutin, and rutin isomers.

Step (e) of the methods described herein involves eluting the adsorbed one or more steviol glycoside stabilizing compounds, and salts thereof, from the weak ion exchange chromatography column to obtain a first eluent comprising one or more steviol glycoside stabilizing compounds, and salts thereof. The eluting is performed under any conditions suitable to elute the one or more steviol glycoside stabilizing compounds, and salts thereof from the column.

An example of suitable conditions to elute the one or more steviol glycoside stabilizing compounds, and salts thereof from the column include eluting the column with any suitable volume of a solution comprising a salt (e.g., sodium chloride, potassium chloride, ammonium chloride, sodium sulfate, potassium sulfate, sodium phosphate, potassium phosphate, and the like). Examples of solutions comprising a salt include solutions comprising at least one salt (e.g., about 5 wt. % to about 25 wt. %, about 15 wt. % to about 20 wt. % or about 5 wt. % to about 10 wt. % of a salt) dissolved in an aqueous composition comprising a (C₁-C₄)alkanol (e.g., at least about 2 BV, at least about 3 BV or at least about 4 BV of an aqueous composition comprising from about 10% to about 60%, about 20% to about 50%, about 30% to about 55%, about 40% to about 60%, or about 50% by volume (C₁-C₄)alkanol).

Another example of suitable conditions to elute the one or more steviol glycoside stabilizing compounds, and salts thereof from the column include eluting the column with any suitable volume of a solution comprising an acid (e.g., hydrochloric acid, sulfuric acid, phosphoric acid, acetic acid, formic acid, and the like). Examples of solutions comprising an acid include solutions comprising hydrochloric acid and the like and optionally acids solutions comprising an aqueous composition comprising from about 10% to about 60%, about 20% to about 50%, about 30% to about 55%, about 40% to about 60%, or about 50% by volume (C₁-C₄)alkanol).

The first eluent comprising the one or more steviol glycoside stabilizing compounds, and salts thereof, collected from the eluting step is collected and can be subsequently concentrated by removing solvent (e.g., to remove water and (C₁-C₄)alkanol) by any suitable means to provide a concentrate comprising the one or more steviol glycoside stabilizing compounds, and salts thereof. The solvent removal can be accomplished under an inert atmosphere (e.g., under a nitrogen gas atmosphere). While not wishing to be bound by any specific theory, it is believed that performing the solvent removal under an inert atmosphere can reduce the formation of highly colored polymeric substances that either natively exist in the yerba mate biomass or form at one or more of the steps described herein.

The first eluent comprising the one or more steviol glycoside stabilizing compounds, and salts thereof comprises a solvent. The solvent can be removed in a step (f) to dryness or it can be removed to a point where a volume of an aqueous composition comprising a (C₁-C₄)alkanol remains as a solvent (e.g., about 50%, about 40%, about 30% about 20%, about 10% or about 5% of an original, total volume of the eluent) to form a concentrate, though the ratio of components that make up the aqueous composition comprising a (C₁-C₄)alkanol may or may not be different from the ratio of components that made up the aqueous composition comprising a (C₁-C₄)alkanol that was used to elute the adsorbed one or more steviol glycoside stabilizing compounds, and salts thereof. Alternatively, the solvent in the eluent comprising the one or more steviol glycoside stabilizing compounds, and salts thereof, can be removed to a point where a volume of an aqueous composition comprising a (C₁-C₄)alkanol remains, wherein the aqueous composition comprising a (C₁-C₄)alkanol comprises less than about 10%, less than about 5%, less than about 2% or less than about 1% by volume (C₁-C₄)alkanol.

Suitable conditions for removing solvent from the eluent comprising, among other of the acids described herein, the one or more steviol glycoside stabilizing compounds, and salts thereof, to form a concentrate comprising, among other of the acids described herein, the one or more steviol glycoside stabilizing compounds, and salts thereof include blowing an inert gas (e.g., nitrogen gas) over the surface of the eluent. The eluent can be heated while blowing the nitrogen gas or it can be at room temperature (e.g., 25° C.). Other conditions for removing the solvent in the eluent include applying a vacuum to the container containing the eluent. The vacuum can be applied with the eluent at room temperature or while heating the container. Yet other conditions for removing solvent in the eluent include passing the eluent through a wiped film evaporator or an agitated thin film evaporator.

The pH of the concentrate can be adjusted at this point to obtain a pH-adjusted concentrate, though adjusting the pH at this point is optional. For example, the pH of the concentrate can be adjusted to a pH where, among other of the acids described herein, the one or more steviol glycoside stabilizing compounds, and salts thereof are protected from degradation. Suitable pHs include pHs of less than about 6, less than about 5, less than about 4, less than about 3 or less than about 2; such as a pH of from about 2 to about 6, about 2 to about 5, about 2 to about 4, about 3 to about 5 or a pH of about 3.5. The pH of the concentrate can be adjusted by using any suitable acid or base. When an acid is used, the acid can be hydrochloric acid and the like.

The concentrate or the pH-adjusted concentrate can be taken on as-is in the methods described herein or the removing step (f) or they can be filtered. The concentrate or the pH-adjusted concentrate can be filtered using any suitable filter (e.g., low ash filter paper, such as Whatman 44 or 54 low ash filter paper), a nylon filter, a polyethersulfone filter, a glass fiber filter, a pad of diatomaceous earth, and the like. In some instances, the pH-adjusted concentrate can be filtered through a polymeric membrane, such as a polyethersulfone (PES) filter having, e.g., 0.2 μm pore size, or a pleated (flat membrane, vacuum filtration) or a pleated PES membrane, depending on the volume of the concentrate or the pH-adjusted concentrate.

The concentrate comprising one or more steviol glycoside stabilizing compounds, and salts thereof, whether it is pH-adjusted, filtered or both pH-adjusted and filtered, can be taken directly to drying step (h) or can be submitted for desalting/decoloring in step (g) (in either order, including desalting, followed by decoloring; decoloring, followed by desalting; decoloring, but not desalting; or desalting, but not decoloring) of a concentrate that can be highly colored. The desalting/decoloring can be accomplished under an inert atmosphere (e.g., under a nitrogen gas atmosphere). While not wishing to be bound by any specific theory, it is believed that performing the one or more steps under an inert atmosphere can reduce the formation of highly colored polymeric substances that either natively exist in the yerba mate biomass or form at one or more of the steps described herein.

The concentrate, whether it is pH-adjusted, filtered or both pH-adjusted and filtered, can be decolored by any suitable means, including ultrafiltration (e.g., filtering through a molecular weight cutoff membrane, size-exclusion chromatography or gel permeation). One obtains a filtrate from decoloring. Ultrafiltration accomplishes, among other things, decoloration of a concentrate that can be highly colored. While not wishing to be bound by any specific theory, it is believed that ultrafiltration removes highly colored polymeric substances that either natively exist in the yerba mate biomass or form at one or more of the steps described herein.

The filtrate from decoloring can be taken on to drying step (h) or it can be desalted in step (g). Alternatively, the concentrate, whether it is pH-adjusted, filtered or both pH-adjusted and filtered, can be desalted without first decoloring. Regardless, the desalting can be accomplished using a nanofiltration membrane and a hydrophobic resin. Those of skill in the art would recognize that when one uses a nanofiltration membrane and a hydrophobic resin one discards the permeate and keeps the retentate. In one example, desalting can be accomplished using a hydrophobic resin (e.g., a porous poly divinylbenzene/ethylvinylbenzene matrix, such as SEPABEADS™ SP70), where one would load a pH-adjusted concentrate (e.g., an acidified concentrate, with a pH of less than about 2) comprising less than about 20% by volume (C₁-C₄)alkanol. The resin is then washed with dilute alcohol (e.g., less than about 10% by volume (C₁-C₄)alkanol, the rest being water having a pH of less than about 2) and then eluted with an aqueous composition comprising about 70% by volume (C₁-C₄)alkanol in water to obtain a desalted second eluent comprising one or more steviol glycoside stabilizing compounds, and salts thereof.

If desalting precedes decoloring in step (g), the solvent in the permeate from the desalting step can be removed to a point where a volume of an aqueous composition comprising a (C₁-C₄)alkanol remains as a solvent (e.g., about 50%, about 40%, about 30% about 20%, about 10% or about 5% of an original, total volume of the eluent) to form a first desalted concentrate. Alternatively, the solvent in the permeate from the desalting can be removed, to give a second desalted concentrate, to a point where a volume of an aqueous composition comprising a (C₁-C₄)alkanol remains, wherein the aqueous composition comprising a (C₁-C₄)alkanol comprises less than about 10%, less than about 5%, less than about 2% or less than about 1% by volume (C₁-C₄)alkanol. The first desalted concentrate can also have the attributes of the second desalted concentrate, such that the first desalted concentrate also has less than about 10%, less than about 5%, less than about 2% or less than about 1% by volume (C₁-C₄)alkanol.

Suitable conditions for removing solvent from the permeate comprising one or more steviol glycoside stabilizing compounds, and salts thereof, to form a first/second desalted concentrate comprising one or more steviol glycoside stabilizing compounds, and salts thereof include blowing an inert gas (e.g., nitrogen gas) over the surface of the eluent. The permeate can be heated while blowing the nitrogen gas or it can be at room temperature (e.g., 25° C.). Other conditions for removing the solvent in the eluent include applying a vacuum to the container containing the permeate. The vacuum can be applied with the permeate at room temperature or while heating the container. Yet other conditions for removing solvent in the permeate include passing the permeate through a wiped film evaporator or an agitated thin film evaporator.

In another example, the concentrate comprising one or more steviol glycoside stabilizing compounds, and salts thereof can be filtered through filter paper to obtain a first filtrate, the first filtrate is ultrafiltered to obtain a second filtrate, and the second filtrate is nanofiltered using a nanofiltration membrane to obtain a first retentate or the second filtrate is eluted through a hydrophobic resin to obtain a desalted second eluent. In another example, the concentrate comprising one or more steviol glycoside stabilizing compounds, and salts thereof can be filtered through filter paper to obtain a first filtrate, the first filtrate is nanofiltered using a nanofiltration membrane to obtain a third retentate or the first filtrate is eluted through a hydrophobic resin to obtain a desalted second eluent, and the third retentate or the desalted second eluent is ultrafiltered to obtain a third filtrate.

As mentioned herein, the eluent comprising one or more steviol glycoside stabilizing compounds, and salts thereof, can be concentrated to dryness or it can be concentrated to a point where a volume of an aqueous composition comprising a (C₁-C₄)alkanol remains. If the eluent is concentrated to dryness, the dry material can be reconstituted using, for example, an aqueous composition comprising a (C₁-C₄)alkanol. The reconstituted material can then be filtered as described herein, to among other things, at least one of desalt and decolor.

The methods described herein can include step (h) that involves drying first retentate, desalted second eluent or the third filtrate to obtain the composition comprising one or more steviol glycoside stabilizing compounds, and salts thereof. The first retentate, desalted second eluent or the third filtrate can be dried in any suitable manner, including by lyophilization or spray drying.

FIG. 1 is a flow diagram of a method 100 for making a composition comprising one or more steviol glycoside stabilizing compounds, and salts thereof. In operation 102, yerba mate biomass is contacted with an aqueous composition containing 50% ethanol/water in a suitable container (e.g., a glass jar) for 1 h (300 g yerba mate biomass into 1.5 L solvent) to obtain an initial extract. In operation 104, the initial extract is filtered using, for example, a ceramic Buchner funnel with Whatman 54 low ash filter paper into glass 4 L side arm flask. In operation 106, the volume of the filtered initial extract is adjusted with an aqueous composition, in this case water, to obtain an adjusted filtered initial extract containing a lower proportion of ethanol, in this case 35% by volume ethanol. In operation 108, the adjusted filtered initial extract can be re-filtered using, for example, a ceramic Buchner funnel with Whatman 44 low ash filter paper into glass 4 L side arm flask. In operation 110, the adjusted filtered initial extract is chromatographed on an ion exchange chromatography stationary phase. For example, AMBERLITE® FPA 53 resin is packed in glass column. The resin is preconditioned with 35% ethanol (2 BV at 2 BV/h). The adjusted filtered initial extract is loaded is loaded (2 BV/h) onto the resin, discarding the loading permeate. The resin is washed with 35% ethanol (4 BV at 2 BV/h) discarding the washing permeate. The one or more steviol glycoside stabilizing compounds, and salts thereof are eluted with 50% ethanol/water, 10% FCC sodium chloride (4 BV, 0.5 BV/h) and the permeate is kept. The column/resin can optionally be regenerated with water (4 BV, 2 BV/h). In operation 112, the eluent/permeate is concentrated to form a concentrate. In this case, nitrogen gas was blown over the top of the eluent/permeate for 2 days, until volume the volume is approximately one third of the initial volume of eluent/permeate and/or ethanol is less than 1% in the eluent/permeate, thereby obtaining a concentrate. In operation 114, the concentrate is acidified to a pH of approximately 3.5 and then filtered through a Whatman 44 filter paper on a Buchner funnel followed by 0.2 μm polyether sulfone (PES) filter. In operation 116, the filtered concentrate is decolored using a molecular weight cutoff membrane (MWCO; e.g., a MWCO membrane that removes materials having a molecular weight of greater than 10 kDA, such as a 3 kDa TURBOCLEAN® NP010 or Synder VT-2B or a 1 kDa Synder XT-2B) to, among other things, decolor the filtered concentrate and obtain a permeate. In operation 118, the permeate is filtered through a nanofiltration membrane (e.g., TRISEP® XN45 membrane) and the retentate is subsequently dried in operation 120 to obtain the composition comprising one or more steviol glycoside stabilizing compounds, and salts thereof.

FIG. 2 is a flow diagram of a method 200 for making a composition comprising one or more steviol glycoside stabilizing compounds, and salts thereof. In operation 202, yerba mate biomass is contacted with an aqueous composition containing 50% ethanol/water in a suitable container (e.g., a glass jar) for 1 h (300 g yerba mate biomass into 1.5 L solvent) to obtain an initial extract. In operation 204, the initial extract is filtered using, for example, a ceramic Buchner funnel with Whatman 54 low ash filter paper into glass 4 L side arm flask. In operation 206, the volume of the filtered initial extract is adjusted with an aqueous composition, in this case water, to obtain an adjusted filtered initial extract containing a lower proportion of ethanol, in this case 35% by volume ethanol. In operation 208, the adjusted filtered initial extract can be re-filtered using, for example, a ceramic Buchner funnel with Whatman 44 low ash filter paper into glass 4 L side arm flask. In operation 210, the adjusted filtered initial extract is chromatographed on an ion exchange chromatography stationary phase. For example, AMBERLITE® FPA 53 resin is packed in glass column. The resin is preconditioned with 35% ethanol (2 BV at 2 BV/h). The adjusted filtered initial extract is loaded is loaded (2 BV/h) onto the resin, discarding the loading permeate. The resin is washed with 35% ethanol (4 BV at 2 BV/h) discarding the washing permeate. The one or more steviol glycoside stabilizing compounds, and salts thereof are eluted with 50% ethanol/water, 10% FCC sodium chloride (4 BV, 0.5 BV/h) and the permeate is kept. The column/resin can optionally be regenerated with water (4 BV, 2 BV/h). In operation 212, the eluent/permeate is concentrated to form a concentrate, where the volume is approximately one third of the initial volume of eluent/permeate and/or ethanol is less than 1% in the eluent/permeate, thereby obtaining a concentrate. In operation 214, the concentrate is acidified to a pH of approximately 1 and then filtered through a Whatman 44 filter paper on a Buchner funnel followed by 0.2 μm polyether sulfone (PES) filter. In operation 218, the concentrate is desalted using a hydrophobic resin (e.g., a porous poly divinylbenzene/ethylvinylbenzene matrix, such as SEPABEADS™ SP70) and the solvent in the retentate is removed in operation 217. In operation 216, the desalted concentrate is decolored using a molecular weight cutoff membrane (MWCO; e.g., a MWCO membrane that removes materials having a molecular weight of greater than 10 kDA, such as a 3 kDa TURBOCLEAN® NP010) to, among other things, decolor the filtered concentrate and obtain a permeate, which is subsequently dried in operation 220 to obtain the composition comprising one or more steviol glycoside stabilizing compounds, and salts thereof.

Another example of a method for making a composition comprising one or more steviol glycoside stabilizing compounds, and salts thereof, the method comprising
(i) contacting yerba mate biomass with an aqueous composition to obtain an initial extract;
(ii) removing solids from the initial extract to obtain a second initial extract;
(iii) contacting the second initial extract with acidified ethyl acetate to obtain an acidic ethyl acetate extract;
(iv) neutralizing the acidic ethyl acetate extract to obtain neutralized ethyl acetate and an aqueous extract;
(v) decoloring the aqueous extract to obtain a decolored aqueous extract; and
(vi) drying the decolored aqueous extract to obtain the composition comprising one or more steviol glycoside stabilizing compounds, and salts thereof.

Steps (i), (ii), and (vi) are performed as described herein for steps (a), (b), and (h). Step (v) is analogous to filtering step (g), except that step (v) involves only decoloring processes, such as ultrafiltration, which includes filtering through a molecular weight cutoff membrane, size-exclusion chromatography, and gel permeation, as discussed herein. Accordingly, the disclosure with regard to steps (a), (b), (g), and (h) applies to steps (i), (ii), (v), and (vi).

Step (i) of the methods described herein involve contacting yerba mate biomass with an aqueous composition to obtain an initial extract comprising one or more steviol glycoside stabilizing compounds, and salts thereof.

The aqueous composition can comprise water and not contain any co-solvents, such as organic solvents. But the aqueous composition can comprise co-solvents, in addition to water. Suitable co-solvents include organic solvents, such as, (C₁-C₄)alkanols and mixtures of (C₁-C₄)alkanols. The proportion of organic solvent, such as (C₁-C₄)alkanol or mixtures of (C₁-C₄)alkanols, can be any suitable proportion such that the aqueous composition can comprise up to about 30%, up to about 40%, up to about 50% or up to about 60% by volume organic solvent, the balance being water; or from about 30% to about 60%, about 40% to about 60%, about 30% to about 50%, about 40% to about 50%, or about 50% by volume organic solvent, the balance being water.

In some instances, the aqueous composition can be buffered with any suitable buffering system, including, but not limited to, a phosphate, citrate, ascorbate, lactate, acetate, and the like. Buffers can be in the range of 1-1000 mM of the anion. Alternatively, water acidified to pH 5-6 with hydrochloric acid, sulfuric acid, nitric acid or the like can be useful in the aqueous composition, with or without a co-solvent. Alternatively, pure water made basic to pH 7-11 with hydroxide, such as sodium or potassium hydroxide can be useful in the aqueous composition, with or without a co-solvent. In still other instances, it may be suitable to add a suitable non-ionic solute that can help balance the osmotic potential of the aqueous composition.

The yerba mate biomass can be stirred, sonicated or otherwise agitated prior to and/or during the contacting of step (i) to, among other things, maximize the extraction of one or more steviol glycoside stabilizing compounds, and salts thereof.

The initial extract can be carried through to step (iii) as-is or bulk solids and or plant solids present, such as comminuted yerba mate plant leaves, stalks, tops, roots, and the like, can be removed in step (ii) of the methods described herein. When step (ii) is carried out, one obtains a second initial extract.

Bulk solids can be removed by any suitable method, including centrifugation, skimming, or filtration. For example, the initial extract can be filtered using any suitable filtration method, including gravity filtration or vacuum filtration through any suitable filter, so long as the filter does not substantially retain the one or more steviol glycoside stabilizing compounds, and salts thereof, including a paper filter (e.g., low ash filter paper, such as Whatman 44 or 54 low ash filter paper), a nylon filter, polyethersulfone filter, a glass fiber filter, a pad of diatomaceous earth, and the like.

Prior to carrying out step (iii) one can optionally adjust the pH of the initial or second initial extract with a suitable acid. (e.g., hydrochloric acid and the like) or suitable base (e.g., sodium hydroxide) to a pH of between about 4 and about 7. The pH-adjusted initial or second initial extract is then extracted with ethyl acetate that has not been pre-acidified as described herein. While not wishing to be bound by any specific theory, it is believed that when the pH of the initial or second initial extract is adjusted to between about 4 and about 7, it is possible to extract certain impurities into the ethyl acetate, while keeping the one or more steviol glycoside stabilizing compounds in the aqueous layer.

Step (iii) of the methods described herein involves contacting the first or second initial extract with acidified ethyl acetate to obtain an acidic ethyl acetate extract. The acidified ethyl acetate can be prepared in any suitable manner, including by adding any suitable acid, including hydrochloric acid, sulfuric acid, and glacial acetic acid (e.g., 0.01-1% vol/vol). The acidic ethyl acetate extract is washed with water (e.g., three times, with 1:1 vol/vol water). Under these conditions, the one or more steviol glycoside stabilizing compounds, and salts thereof, will substantially be in their conjugate acid form and will reside substantially in the acidic ethyl acetate layer that forms when the acidic ethyl acetate extract is washed with water. The water layers are discarded and the acidic ethyl acetate extract is carried on to step (iv).

Step (iii) of the methods described herein can be carried out in other suitable ways, including by using ethyl acetate that has not been pre-acidified as described herein (e.g., by pre-washing with glacial acetic acid), but instead by adjusting the pH of the initial or second initial extract with a suitable acid. (e.g., hydrochloric acid and the like), then extracting the pH-adjusted initial or second initial extract with ethyl acetate that has not been pre-acidified. Regardless of the acid used to adjust the pH of the initial extract or the second initial extract, the pH of the initial extract or the second initial extract is adjusted to about 4 or less, 3 or less, about 2 or less, or about 1 or less. The water layers are discarded and the acidic ethyl acetate extract that results is carried on to step (iv).

Step (iv) of the methods described herein involves neutralizing the acidic ethyl acetate extract to obtain neutralized ethyl acetate and an aqueous extract. This is accomplished in any suitable way, including washing the acidic ethyl acetate extract with water (e.g., three times, with 1:1 vol/vol water) comprising a suitable base, such as sodium hydroxide, potassium hydroxide, and the like, and combinations thereof. Under these conditions, the one or more steviol glycoside stabilizing compounds, and salts thereof, will substantially be in their conjugate base form and will substantially reside in the water layer that forms when the acidic ethyl acetate extract is washed with water comprising a suitable base.

In an alternative, optional step to step (iv), step (iv-a), the acidic ethyl acetate extract that results from step (iii) can be optionally removed, even removed to dryness. Any solid that remains can either be reconstituted with pH neutral water (e.g., deionized water) and the pH of the water can then be adjusted to about 3 to about 7; or the solid that remains can be reconstituted with water having a pH of about 3 to about 7.

The aqueous extract comprising the one or more steviol glycoside stabilizing compounds, and salts thereof, whether they emanate from step (iv) or step (iv-a), can then be submitted for step (v) to accomplish, among other things, decoloring of aqueous extract, which can be highly colored. Decoloring can be accomplished by any suitable means, including ultrafiltration (e.g., filtering through a molecular weight cutoff membrane, size-exclusion chromatography, or gel permeation). One obtains a filtrate from decoloring. Ultrafiltration accomplishes, among other things, decoloration of a concentrate that can be highly colored. While not wishing to be bound by any specific theory, it is believed that ultrafiltration removes highly colored polymeric substances that either natively exist in the yerba mate biomass or form at one or more of the steps described herein.

Another example of modifications to the method described herein comprising steps (i)-(vi) (including the alternative, optional step (iv-a) includes a method for making a composition comprising one or more steviol glycoside stabilizing compounds, and salts thereof, the method comprising
contacting yerba mate biomass with an aqueous composition to obtain an initial extract;
removing solids from the initial extract to obtain a second initial extract;
adjusting the pH of the second initial extract to a pH of from about 4 to about 7 to obtain a first pH-adjusted second initial extract;
contacting the first pH-adjusted second initial extract with ethyl acetate to obtain a first ethyl acetate extract and a second aqueous extract;
adjusting the pH of the second aqueous extract to a pH of less than 2 to obtain a pH-adjusted second aqueous extract;
contacting the pH-adjusted second aqueous extract with ethyl acetate to obtain a second ethyl acetate extract;
removing the ethyl acetate from the second ethyl acetate extract to obtain a purified composition; reconstituting the crude composition with water to obtain a third aqueous extract; and
decoloring the third aqueous extract to obtain a decolored aqueous extract. The “purified composition” will comprise the compounds of interest (e.g., the one or more steviol glycoside stabilizing compounds, and salts thereof) and is purified relative to at least the initial extract and the second initial extract, in that the “purified composition” will not contain certain impurities in the initial extract and the second initial extract, but does contain highly colored polymeric substances that either natively exist in the yerba mate biomass or form at one or more of the steps described herein and that are removed in the decoloring step.

Yet another example of modifications to the method described herein comprising steps (i)-(vi) (including the alternative, optional step (iv-a) includes a method for making a composition comprising one or more steviol glycoside stabilizing compounds, and salts thereof, the method comprising
contacting yerba mate biomass with an aqueous composition to obtain an initial extract;
removing solids from the initial extract to obtain a second initial extract;
adjusting the pH of the second initial extract to a pH of less than about 2 to obtain a second pH-adjusted second initial extract;
contacting the second pH-adjusted second initial extract with ethyl acetate to obtain a third ethyl acetate extract;
neutralizing the third ethyl acetate extract to obtain a first neutralized ethyl acetate extract and a third aqueous extract; and
decoloring the third aqueous extract to obtain a decolored aqueous extract.

The methods described herein can include step (vi) that involves drying the decolored aqueous extract to obtain the composition comprising one or more steviol glycoside stabilizing compounds, and salts thereof. The first or second retentates or the third filtrate can be dried in any suitable manner, including by lyophilization or spray drying.

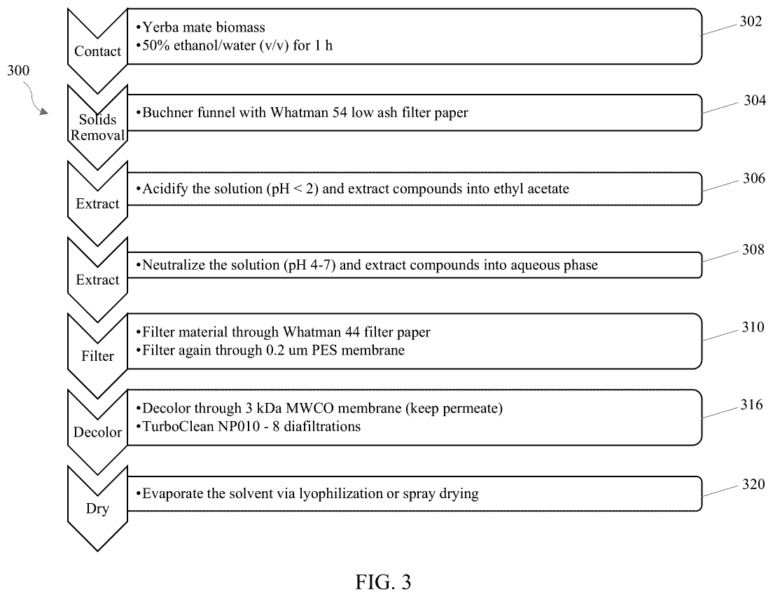

FIG. 3 is a flow diagram of a method 300 for making a composition comprising one or more steviol glycoside stabilizing compounds, and salts thereof. In operation 302, yerba mate biomass is contacted with an aqueous composition containing 50% ethanol/water in a suitable container (e.g., a glass jar) for 1 h (300 g yerba mate biomass into 1.5 L solvent) to obtain an initial extract. In operation 304, the initial extract is filtered using, for example, a ceramic Buchner funnel with Whatman 54 low ash filter paper into glass 4 L side arm flask to, among other things, remove solids from, e.g., the yerba mate biomass. The filtrate from operation 304 is extracted in operation 306 with acidified ethyl acetate extraction. Following extraction of the one or more steviol glycoside stabilizing compounds into the acidified ethyl acetate, the acidified ethyl acetate is washed with water comprising a suitable base, such as sodium hydroxide, potassium hydroxide, and the like, in operation 308 to obtain neutralized ethyl acetate and an aqueous extract. Under these conditions, the one or more steviol glycoside stabilizing compounds will substantially be in their conjugate base form and will substantially reside in the water layer that forms when the acidic ethyl acetate extract is washed with water comprising a suitable base. In operation 310 the water layer is filtered to obtain a filtrate. In operation 316, the filtrate is decolored using a 3 kDa molecular weight cutoff membrane (TURBOCLEAN® NP010; six diafiltrations) to, among other things, decolor the aqueous extract, thereby obtaining a decolored aqueous extract. In operation 320, the decolored aqueous extract is dried to obtain the composition comprising one or more steviol glycoside stabilizing compounds, and salts thereof.

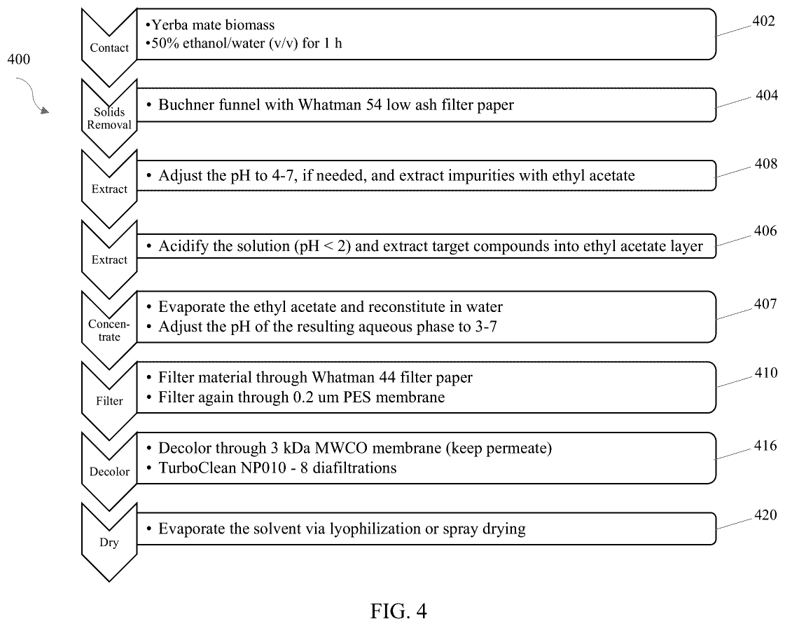

FIG. 4 is a flow diagram of a method 400 for making a composition comprising one or more steviol glycoside stabilizing compounds, and salts thereof. In operation 402, yerba mate biomass is contacted with an aqueous composition containing 50% ethanol/water in a suitable container (e.g., a glass jar) for 1 h (300 g yerba mate biomass into 1.5 L solvent) to obtain an initial extract. In operation 404, the initial extract is filtered using, for example, a ceramic Buchner funnel with Whatman 54 low ash filter paper into glass 4 L side arm flask to, among other things, remove solids from, e.g., the yerba mate biomass. The filtrate from operation 404 is pH-adjusted to from about 4 to about 7 and the filtrate is extracted in operation 408 with ethyl acetate, while the compounds of interest remain in the aqueous layer. In operation 406, the pH of the aqueous layer is adjusted to less than 2 and the aqueous layer is extracted with ethyl acetate. Following extraction of the one or more steviol glycoside stabilizing compounds into the ethyl acetate, the ethyl acetate is removed to dryness in operation 407 to obtain a solid. The solid is reconstituted with water and the pH of the water is adjusted to from about 3 to about 7. Under these conditions, the one or more steviol glycoside stabilizing compounds will substantially be in their conjugate base form and will dissolve in the water. In operation 410 the water layer is filtered to obtain a filtrate. In operation 416, the filtrate is decolored using a 3 kDa molecular weight cutoff membrane (TURBOCLEAN® NP010; six diafiltrations) to, among other things, decolor the aqueous extract, thereby obtaining a decolored aqueous extract. In operation 420, the decolored aqueous extract is dried to obtain the composition comprising one or more steviol glycoside stabilizing compounds, and salts thereof.

The composition comprising the one or more steviol glycoside stabilizing compounds, and salts thereof prepared according to the methods described herein can be incorporated into any ingestible composition, including into beverages and food products.

For example, the ingestible composition can be a comestible composition or noncomestible composition. By “comestible composition”, it is meant any composition that can be consumed as food by humans or animals, including solids, gel, paste, foamy material, semi-solids, liquids, or mixtures thereof. By “noncomestible composition”, it is meant any composition that is intended to be consumed or used by humans or animals not as food, including solids, gel, paste, foamy material, semi-solids, liquids, or mixtures thereof. The noncomestible composition includes, but is not limited to medical compositions, which refers to a noncomestible composition intended to be used by humans or animals for therapeutic purposes. By “animal”, it includes any non-human animal, such as, for example, farm animals and pets.

The composition comprising the one or more steviol glycoside stabilizing compounds, and salts thereof prepared according to the methods described herein can be added to a noncomestible composition or non-edible product, such as supplements, nutraceuticals, functional food products (e.g., any fresh or processed food claimed to have a health-promoting and/or disease-preventing properties beyond the basic nutritional function of supplying nutrients), pharmaceutical and over the counter medications, oral care products such as dentifrices and mouthwashes, cosmetic products such as lip balms and other personal care products.

In general, over the counter (OTC) product and oral hygiene product generally refer to product for household and/or personal use which may be sold without a prescription and/or without a visit to a medical professional. Examples of the OTC products include, but are not limited to Vitamins and dietary supplements; Topical analgesics and/or anaesthetic; Cough, cold and allergy remedies; Antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anaesthetic include any topical creams/ointments/gels used to alleviate superficial or deep-seated aches and pains, e.g., muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral hygiene products include, but are not limited to mouth cleaning strips, toothpaste, toothbrushes, mouthwashes/dental rinses, denture care, mouth fresheners at-home teeth whiteners and dental floss.

The composition comprising one or more steviol glycoside stabilizing compounds, and salts thereof prepared according to the methods described herein can be added to food or beverage products or formulations. Examples of food and beverage products or formulations include, but are not limited to coatings, frostings, or glazes for comestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready to serve, semi condensed or condensed and may be served hot or cold, as a first course or as the main course of a meal or as a between meal snack (sipped like a beverage). Soup may be used as an ingredient for preparing other meal components and may range from broths ( consommé) to sauces (cream or cheese based soups).

“Dehydrated and Culinary Food Category” usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready to cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category means beverages, beverage mixes and concentrates, including but not limited to, carbonated and non-carbonated beverages, alcoholic and nonalcoholic beverages, ready to drink beverages, liquid concentrate formulations for preparing beverages such as sodas, and dry powdered beverage precursor mixes. The Beverage category also include the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The hot drinks include, but are not limited to coffee, such as fresh (e.g., brewed), instant, combined coffee, liquid, ready-to-drink, soluble and dry coffee beverages, coffee beverage mixes and concentrates (syrups, pure, formulated, or in powder form; example of a “powder form” is a product comprising coffee, sweetener, and whitener all in powder form); tea, such as black, green, white, oolong, and flavored tea; and other hot drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionary category generally refers to edible product that is sweet to the taste. Examples of confectionary include, but are not limited to candies, gelatins, chocolate confectionery, sugar confectionery, gum, and the likes and any combination products. The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The read meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles.

The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for comestible composition, particularly food and beverage products or formulations, are provided as follows. Exemplary comestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarized gum, sugar free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi pack dairy ice cream, multi pack water ice cream, take home ice cream, take home dairy ice cream, ice cream desserts, bulk ice cream, take home water ice cream, frozen yoghurt, artisanal ice cream, dairy products, milk, fresh/pasteurized milk, full fat fresh/pasteurized milk, semi skimmed fresh/pasteurized milk, long life/uht milk, full fat long life/uht milk, semi skimmed long life/uht milk, fat free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners (e.g., dairy and non-dairy based creamers or whiteners for coffee beverages), powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf stable desserts, dairy based desserts, soy based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars; energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow on milk formula, toddler milk formula, hypoallergenic milk formula, prepared baby food, dried baby food, other baby food, spreads, jams and preserves, honey, chocolate spreads, nut based spreads, and yeast-based spreads. Examples of comestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Examples of comestible compositions also include breakfast cereals, sweet beverages or solid or liquid concentrate compositions for preparing beverages. Examples of comestible compositions also include coffee flavored food (e.g., coffee flavored ice cream).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

### EXAMPLES

The present invention can be better understood by reference to the following examples which are offered by way of illustration. The present invention is not limited to the examples given herein.

### Example 1

Samples were prepared via the design shown in Table 1, in weight to volume percentage. An appropriate amount of steviol glycoside (SG) was weighed into a 10 mL glass vial and diluted with an appropriate volume of pH 4 citrate buffer, e.g., for 0.6% level, 27 mg was diluted into 4.5 mL of buffer. This was repeated for all conditions in Table 1. Samples that were designed for pH 2.5 were then adjusted via phosphoric acid and pH meter to pH 2.5, dropwise. For these samples, the same lot of steviol glycoside stabilizing compound (SC) was used, which was purified from stevia leaves. Two different SG sources were used, RM80 (>80% Reb M on a dry weight basis) and RA95 (>95% Reb A on a dry weight basis).

At each time point, the solutions were centrifuged at 10,000 rpm for two minutes to remove any insoluble material from the analysis (even though none was visible). An aliquot of the supernatant was diluted into 55% methanol for analysis by UHPLC-UV. The chromatographic analysis was performed on a C18-based reversed-phase chromatography column at elevated temperature under gradient conditions, utilizing trifluoroacetic acid in water and acetonitrile. SGs were detected utilizing a UV detector set to 210 nm. A linear calibration curve was applied using a high-purity (>99%) Reb A standard as a reference solution.

TABLE 1

| SG % | SG Type | SC % | Storage Cond | pH | Times (weeks) | # of Pulls | # of Replicates |
|---|---|---|---|---|---|---|---|
| 0.6% | RM80 | 0.6% | RT | 2.5 | 0, 14, 26, 52 | 4 | 3 |
| 1.5% | RM80 | 1.5% | RT | 2.5 | 0, 4, 14, 26, 39, 52 | 6 | 1 |
| 3.0% | RM80 | 3.0% | RT | 2.5 | 0, 4, 14, 26, 39, 52 | 6 | 3 |
| 3.0% | RM80 | 4.5% | RT | 2.5 | 0, 4, 14, 26, 39, 52 | 6 | 3 |
| 6.0% | RM80 | 6.0% | RT | 2.5 | 0, 4, 14, 26, 39, 52 | 6 | 1 |
| 6.0% | RA95 | 6.0% | RT | 2.5 | 0, 4, 14, 26, 39, 52 | 6 | 3 |
| 0.6% | RM80 | 0.6% | 4C | 2.5 | 14, 26, 52 | 3 | 1 |
| 1.5% | RM80 | 1.5% | 4C | 2.5 | 4, 14, 26, 39, 52 | 5 | 3 |
| 3.0% | RM80 | 3.0% | 4C | 2.5 | 4, 14, 26, 39, 52 | 5 | 1 |
| 3.0% | RM80 | 4.5% | 4C | 2.5 | 4, 14, 26, 39, 52 | 5 | 1 |
| 6.0% | RM80 | 6.0% | 4C | 2.5 | 4, 14, 26, 39, 52 | 5 | 3 |
| 6.0% | RA95 | 6.0% | 4C | 2.5 | 4, 14, 26, 39, 52 | 5 | 1 |
| 0.6% | RM80 | 0.6% | RT | 4.0 | 0, 14, 26, 52 | 4 | 3 |
| 1.5% | RM80 | 1.5% | RT | 4.0 | 0, 4, 14, 26, 39, 52 | 6 | 1 |
| 3.0% | RM80 | 3.0% | RT | 4.0 | 0, 4, 14, 26, 39, 52 | 6 | 3 |
| 3.0% | RM80 | 4.5% | RT | 4.0 | 0, 4, 14, 26, 39, 52 | 6 | 3 |
| 6.0% | RM80 | 6.0% | RT | 4.0 | 0, 4, 14, 26, 39, 52 | 6 | 1 |
| 35.0% | RM80 | 35.0% | RT | 4.0 | 0, 26, 40, 52 | 4 | 1 |
| 6.0% | RA95 | 6.0% | RT | 4.0 | 0, 4, 14, 26, 39, 52 | 6 | 3 |
| 0.6% | RM80 | 0.6% | 4C | 4.0 | 14, 26, 52 | 3 | 1 |
| 1.5% | RM80 | 1.5% | 4C | 4.0 | 4, 14, 26, 39, 52 | 5 | 3 |
| 3.0% | RM80 | 3.0% | 4C | 4.0 | 4, 14, 26, 39, 52 | 5 | 1 |
| 3.0% | RM80 | 4.5% | 4C | 4.0 | 4, 14, 26, 39, 52 | 5 | 1 |
| 6.0% | RM80 | 6.0% | 4C | 4.0 | 4, 14, 26, 39, 52 | 5 | 3 |
| 6.0% | RA95 | 6.0% | 4C | 4.0 | 4, 14, 26, 39, 52 | 5 | 1 |

Briefly, long term storage chemical stability data stored at 4° C.; room temperature (˜22° C.); at pH 4; and at pH 2.5>94% recovery of the SG after 48+ weeks of storage. The long term storage chemical stability data is given in Table 2. A value of NM denotes that no measurement was taken at that time.

TABLE 2

| | Time | | | | | | | |
|---|---|---|---|---|---|---|---|---|
| Experiment | (weeks) | 0 | 4 | 14 | 26 | 39 | 40 | 48 |
| 6% RA95 | Reb A % | 100.0 | 99.5 | 98.3 | 98.3 | 98.6 | NM | 98.5 |
| with 6% | Recovery | | | | | | | |
| SCs at 4 C. | | | | | | | | |
| and pH 2.5 | | | | | | | | |
| 6% RA95 | Reb A % | 100.0 | 98.8 | 99.1 | 98.0 | 98.7 | NM | 98.6 |
| with 6% | Recovery | | | | | | | |
| SCs at 4 C. | | | | | | | | |
| and pH 4 | | | | | | | | |
| 6% RA95 | Reb A % | 100.0 | 99.5 | 98.6 | 98.2 | 98.0 | NM | 97.8 |
| with 6% | Recovery | | | | | | | |
| SCs at RT | | | | | | | | |
| and pH 2.5 | | | | | | | | |
| 6% RA95 | Reb A % | 100.0 | 99.4 | 98.4 | 98.4 | 98.3 | NM | 98.3 |
| with 6% | Recovery | | | | | | | |
| SCs at RT | | | | | | | | |
| and pH 4 | | | | | | | | |
| 0.6% | Reb M % | 100.0 | NM | 102.4 | 102.4 | NM | NM | 98.0 |
| RM80 with | Recovery | | | | | | | |
| 0.6% SCs | | | | | | | | |
| at 4 C. and | | | | | | | | |
| pH 2.5 | | | | | | | | |
| 0.6% | Reb M % | 100.0 | NM | 102.8 | 102.6 | NM | NM | 99.0 |
| RM80 with | Recovery | | | | | | | |
| 0.6% SCs | | | | | | | | |
| at 4 C. and | | | | | | | | |
| pH 4 | | | | | | | | |
| 0.6% | Reb M % | 100.0 | NM | 101.1 | 100.2 | NM | NM | 96.0 |
| RM80 with | Recovery | | | | | | | |
| 0.6% SCs | | | | | | | | |
| at RT and | | | | | | | | |
| pH 2.5 | | | | | | | | |
| 0.6% | Reb M % | 100.0 | NM | 101.7 | 101.5 | NM | NM | 99.2 |
| RM80 with | Recovery | | | | | | | |
| 0.6% SCs | | | | | | | | |
| at RT and | | | | | | | | |
| pH 4 | | | | | | | | |
| 1.5% | Reb M % | 100.0 | 102.6 | 102.6 | 102.5 | 98.5 | NM | 97.9 |
| RM80 with | Recovery | | | | | | | |
| 1.5% SCs | | | | | | | | |
| at 4 C. and | | | | | | | | |
| pH 2.5 | | | | | | | | |
| 1.5% | Reb M % | 100.0 | 102.7 | 102.5 | 102.9 | 98.8 | NM | 98.7 |
| RM80 with | Recovery | | | | | | | |
| 1.5% SCs | | | | | | | | |
| at 4 C. and | | | | | | | | |
| pH 4 | | | | | | | | |
| 1.5% | Reb M % | 100.0 | 101.8 | 100.8 | 98.6 | 97.3 | NM | 95.3 |
| RM80 with | Recovery | | | | | | | |
| 1.5% SCs | | | | | | | | |
| at RT and | | | | | | | | |
| pH 2.5 | | | | | | | | |
| 1.5% | Reb M % | 100.0 | 102.1 | 101.5 | 101.8 | 99.2 | NM | 98.3 |
| RM80 with | Recovery | | | | | | | |
| 1.5% SCs | | | | | | | | |
| at RT and | | | | | | | | |
| pH 4 | | | | | | | | |
| 3% RM80 | Reb M % | 100.0 | 102.5 | 102.2 | 102.1 | 98.5 | NM | 97.9 |
| with 3% | Recovery | | | | | | | |
| SCs at 4 C. | | | | | | | | |
| and pH 2.5 | | | | | | | | |
| 3% RM80 | Reb M % | 100.0 | 102.6 | 102.1 | 102.5 | 98.9 | NM | 98.5 |
| with 3% | Recovery | | | | | | | |
| SCs at 4 C. | | | | | | | | |
| and pH 4 | | | | | | | | |
| 3% RM80 | Reb M % | 100.0 | 101.8 | 101.0 | 99.8 | 95.9 | NM | 95.0 |
| with 3% | Recovery | | | | | | | |
| SCs at RT | | | | | | | | |
| and pH 2.5 | | | | | | | | |
| 3% RM80 | Reb M % | 100.0 | 102.0 | 101.6 | 101.2 | 98.2 | NM | 97.7 |
| with 3% | Recovery | | | | | | | |
| SCs at RT | | | | | | | | |
| and pH 4 | | | | | | | | |
| 3% RM80 | Reb M % | 100.0 | 103.5 | 103.4 | 103.3 | 98.3 | NM | 96.7 |
| with 4.5% | Recovery | | | | | | | |
| SCs at 4 C. | | | | | | | | |
| and pH 2.5 | | | | | | | | |
| 3% RM80 | Reb M % | 100.0 | 103.4 | 102.8 | 103.6 | 97.4 | NM | 97.7 |
| with 4.5% | Recovery | | | | | | | |
| SCs at 4 C. | | | | | | | | |
| and pH 4 | | | | | | | | |
| 3% RM80 | Reb M % | 100.0 | 102.7 | 101.5 | 100.6 | 96.2 | NM | 94.0 |
| with 4.5% | Recovery | | | | | | | |
| SCs at RT | | | | | | | | |
| and pH 2.5 | | | | | | | | |
| 3% RM80 | Reb M % | 100.0 | 103.4 | 102.3 | 102.2 | 98.0 | NM | 97.7 |
| with 4.5% | Recovery | | | | | | | |
| SCs at RT | | | | | | | | |
| and pH 4 | | | | | | | | |
| 6% RM80 | Reb M % | 100.0 | 102.0 | 102.0 | 101.8 | 97.9 | NM | 97.4 |
| with 6% | Recovery | | | | | | | |
| SCs at 4 C. | | | | | | | | |
| and pH 2.5 | | | | | | | | |
| 6% RM80 | Reb M % | 100.0 | 102.0 | 101.6 | 102.2 | 98.3 | NM | 97.7 |
| with 6% | Recovery | | | | | | | |
| SCs at 4 C. | | | | | | | | |
| and pH 4 | | | | | | | | |
| 6% RM80 | Reb M % | 100.0 | 101.6 | 100.7 | 99.5 | 95.0 | NM | 94.1 |
| with 6% | Recovery | | | | | | | |
| SCs at RT | | | | | | | | |
| and pH 2.5 | | | | | | | | |
| 6% RM80 | Reb M % | 100.0 | 101.6 | 101.2 | 100.8 | 97.2 | NM | 96.7 |
| with 6% | Recovery | | | | | | | |
| SCs at RT | | | | | | | | |
| and pH 4 | | | | | | | | |
| 35% RM80 | Reb M % | 100.0 | NM | NM | 99.5 | NM | 95.9 | 96.2 |
| with 35% | Recovery | | | | | | | |
| SCs at RT | | | | | | | | |
| and pH 4 | | | | | | | | |

This example shows that the steviol glycoside stability compounds are effective to stabilize steviol glycoside over time. Steviol glycoside stability compounds are effective to stabilize steviol glycoside over 48 weeks with greater than 94% recovery of the steviol glycoside at 4° C., at room temperature, at pH 4, and/or at pH 2.5.

### Example 2

Samples were prepared at 500 ppm (a use level that can be used in beverages) for storage at room temperature (RT, which is ˜22° C.). A pH 1.7 buffer (Oakton, part number 00654-01) was purchased from Fisher and used to dilute all samples. The lot of SC material used in the study was derived from yerba mate. Three total solutions were made for the study: one with no additives (500 ppm Reb M in pH 1.7 buffer), one with 500 ppm Reb M and 500 ppm SCs, and one negative control with 500 ppm Reb M and 500 ppm ascorbic acid (a common antioxidant).

Highly purified Reb M (>99%) was weighed directly into a 40 mL glass vial at an appropriate level, so the final concentration was 500 ppm, i.e., 20 mg into 40 mL. Next the additive (if present) was weighed directly in the same vial. Finally the total volume of pH 1.7 buffer was added and the solution was mixed to dissolve.

At each time point, the solutions were centrifuged at 10,000 rpm for two minutes to remove any insoluble material from the analysis, even though none was visible. An aliquot of the supernatant was injected directly for analysis by UHPLC-UV. The chromatographic analysis was performed on a C18-based reversed-phase chromatography column at elevated temperature under gradient conditions, utilizing trifluoroacetic acid in water and acetonitrile. SGs were detected utilizing a UV detector set to 210 nm. A linear calibration curve was applied using a high-purity (>99%) Reb A standard as a reference solution.

The results presented in Table 3 demonstrate that SCs generally improve the chemical stability of SGs, even at relatively low levels, such as at the use level.

TABLE 3

| Experiment | Time (Days) | 0 | 7 | 14 | 24 | 35 |
|---|---|---|---|---|---|---|
| Reb M pH 1.7 | % Reb M | 100.0 | 93.4 | 87.7 | 80.2 | 73.0 |
| | Recovery | | | | | |
| Reb M pH 1.7 | % Reb M | 100.0 | 93.6 | 88.0 | 80.5 | 73.5 |
| with AA | Recovery | | | | | |
| Reb M pH 1.7 | % Reb M | 100.0 | 94.0 | 88.7 | 81.4 | 74.6 |
| with SCs | Recovery | | | | | |

### Example 3

Twelve total solutions were made for this study: 0.1% RA95, RM80, or Reb M with no additives; 0.1% RA95, RM80, or Reb M with 0.1% SCs; 1% RA95, RM80, or Reb M with 1% SCs; and 5% RA95, RM80, or Reb M with 5% SCs. To prepare each solution RA95, RM80, or highly purified Reb M (>99%) was weighed into a glass vial in the appropriate amount followed by SC material (derived from yerba mate). Samples were diluted in water and heated at 80° C. to solubilize the glycosides. After allowing solutions to cool to room temperature, phosphoric acid was added to give a final concentration of 5% phosphoric acid. Samples were stored at 40° C. for the duration of the study.

At each time point, the solutions were centrifuged at 10,000 rpm for two minutes to remove any insoluble material from the analysis (even though none was visible). An aliquot of the supernatant was diluted into water for analysis by UHPLC-UV. The chromatographic analysis was performed on a C18-based reversed-phase chromatography column at elevated temperature under gradient conditions, utilizing trifluoroacetic acid in water and acetonitrile. SGs were detected utilizing a UV detector set to 210 nm. A linear calibration curve was applied using a high-purity (>99%) Reb A standard as a reference solution. Table 4 shows the percent recovery data from RA95 (>99%) study in 5% phosphoric acid matrix (pH<<1) stored at 40° C. And Table 5 shows the percent recovery data from pure Reb M (>99%) study in 5% phosphoric acid matrix (pH<<1) stored at 40° C. Finally, Table 6 shows the percent recovery data from Reb M in RM80 product in 5% phosphoric acid matrix (pH<<1) stored at 40° C.

TABLE 4

| Experiment | Time (Days) | 0 | 1 | 2 | 3 | 7 |
|---|---|---|---|---|---|---|
| 0.1% RA95 No | % Reb A | 100.0 | 57.7 | 34.4 | 22.2 | 6.58 |
| SCs | Recovery | | | | | |
| 0.1% RA95 0.1% | % Reb A | 100.0 | 58.3 | 35.6 | 23.1 | 6.68 |
| SCs | Recovery | | | | | |
| 1% RA95 1% | % Reb A | 100.0 | 73.8 | 55.5 | 42.6 | 16.2 |
| SCs | Recovery | | | | | |
| 5% RA95 5% | % Reb A | 100.0 | 89.7 | 81.1 | 73.4 | 50.0 |
| SCs | Recovery | | | | | |

TABLE 5

| Experiment | Time (Days) | 0 | 1 | 2 | 3 | 7 |
|---|---|---|---|---|---|---|
| 0.1% Reb M No | % Reb M | 100.0 | 55.1 | 31.5 | 19.9 | 5.07 |
| SCs | Recovery | | | | | |
| 0.1% Reb M | % Reb M | 100.0 | 57.9 | 34.2 | 21.9 | 6.28 |
| 0.1% SCs | Recovery | | | | | |
| 1% Reb M 1% | % Reb M | 100.0 | 71.8 | 51.9 | 38.5 | 13.4 |
| SCs | Recovery | | | | | |
| 5% Reb M 5% | % Reb M | 100.0 | 85.3 | 73.0 | 62.9 | 36.1 |
| SCs | Recovery | | | | | |

TABLE 6

| Experiment | Time (Days) | 0 | 1 | 2 | 3 | 7 |
|---|---|---|---|---|---|---|
| 0.1% RM80 with 0% | % Reb M | 100.0 | 60.1 | 35.8 | 22.1 | 4.36 |
| SCs and 5% H3PO4 | Recovery | | | | | |
| 0.1% RM80 with | % Reb M | 100.0 | 62.2 | 37.9 | 24.1 | 5.07 |
| 0.1% SCs and 5% | Recovery | | | | | |
| H3PO4 | | | | | | |
| 1% RM80 with 1% | % Reb M | 100.0 | 77.0 | 58.3 | 45.4 | 17.0 |
| SCs and 5% H3PO4 | Recovery | | | | | |
| 5% RM80 with 5% | % Reb M | 100.0 | 91.2 | 83.0 | 75.2 | 49.4 |
| SCs and 5% H3PO4 | Recovery | | | | | |

The results in Tables 4-6 demonstrate that the steviol glycoside stability is concentration-dependent. That is, the higher the concentration of steviol glycoside (SG) and steviol glycoside stabilizing compound, the more stable the steviol glycoside. Thus, for example, at 5 wt. % steviol glycoside and 5 wt. % steviol glycoside stabilizing compound, one observes a significant increase in the stability of the SG as compared to a lower concentration solution.

### Example 4

Three solutions were made for this study: 0.1% RM80 with no additives, 0.1% RM80 with 0.1% SCs, and 0.1% RM80 with 0.3% SCs. To prepare each solution, RM80 was weighed into a glass vial in the appropriate amount followed by SC material (derived from yerba mate). Samples were diluted in water and vortexed to solubilize the glycosides. Phosphoric acid was added to give a final concentration of 0.1% phosphoric acid. Samples were stored at room temperature (20-24° C.) for the duration of the study.

At each time point, the solutions were centrifuged at 10,000 rpm for two minutes to remove any insoluble material from the analysis (even though none was visible). An aliquot of the supernatant was diluted into water for analysis by UHPLC-UV. The chromatographic analysis was performed on a C18-based reversed-phase chromatography column at elevated temperature under gradient conditions, utilizing trifluoroacetic acid in water and acetonitrile. SGs were detected utilizing a UV detector set to 210 nm. A linear calibration curve was applied using a high-purity (>99%) Reb A standard as a reference solution. Table 7 shows data from a use level (e.g., 0.1% RM80) study in 0.1% phosphoric acid matrix at room temperature (˜22° C.). Table 8 is the statistical evaluation of the stability enhancement at each timepoint compared to the solution without stability enhancing compounds.

TABLE 7

| | Time | | | | | | | | |
|---|---|---|---|---|---|---|---|---|---|
| Experiment | (Days) | 0 | 9 | 16 | 30 | 37 | 51 | 58 | 65 |
| 0.1% RM80 witout SCs | % Reb M | 100.0 | 97.8 | 96.6 | 93.6 | 91.5 | 89.6 | 88.1 | 86.2 |
| and 0.1% H3PO4 | Recovery | | | | | | | | |
| 0.1% RM80 with 0.1% | % Reb M | 100.0 | 99.6 | 98.0 | 94.5 | 93.5 | 91.4 | 90.9 | 90.1 |
| SCs and 0.1% H3PO4 | Recovery | | | | | | | | |
| 0.1% RM80 with 0.3% | % Reb M | 100.0 | 98.9 | 97.5 | 94.0 | 93.3 | 91.0 | 91.1 | 89.9 |
| SCs and 0.1% H3PO4 | Recovery | | | | | | | | |

TABLE 8

| | Time | | | | | | | | |
|---|---|---|---|---|---|---|---|---|---|
| Experiment | (Days) | 0 | 9 | 16 | 30 | 37 | 51 | 58 | 65 |
| 0.1% RM80 witout SCs | % Reb M | — | — | — | — | — | — | — | — |
| and 0.1% H3PO4 | Recovery | | | | | | | | |
| 0.1% RM80 with 0.1% | % Reb M | — | 0.0002 | 0.004 | 0.02 | 0.01 | 0.002 | 0.004 | 0.0003 |
| SCs and 0.1% H3PO4 | Recovery | | | | | | | | |
| 0.1% RM80 with 0.3% | % Reb M | — | 0.01 | 0.02 | 0.3 | 0.001 | 0.06 | 0.002 | 0.0009 |
| SCs and 0.1% H3PO4 | Recovery | | | | | | | | |

p-values comparing % reb M remaining as compared to the respective timepoint without SCs present.

These results demonstrate that even at use levels, SCs provide SGs protection from acid hydrolysis. The results also demonstrate that additional SCs beyond the concentration needed to complex all of the SGs can have no additional benefit. For example, 0.3% SCs has the same protective effects as 0.1% SCs.

### Example 5

Four solutions were made for this study using Rosmarinic Acid: 0.1% Reb M with no additives; 0.1% Reb M with 0.1% SCs; 1% Reb M with 1% SCs; and 5% Reb M with 5% SCs. Four solutions were made for this study using Cichoric Acid: 0.1% Reb M with no additives; 0.1% Reb M with 0.1% SCs; 1% Reb M with 1% SCs; and 5% Reb M with 5% SCs. To prepare each solution highly purified Reb M (>99%) was weighed into a glass vial in the appropriate amount followed by SC material (either Rosmarinic Acid or Cichoric Acid). Samples were diluted in water and heated at 80° C. to solubilize the glycosides. After allowing solutions to cool to room temperature, phosphoric acid was added to give a final concentration of 5% phosphoric acid. Samples were stored at 40° C. for the duration of the study.

At each time point, the solutions were centrifuged at 10,000 rpm for two minutes to remove any insoluble material from the analysis (even though none was visible). An aliquot of the supernatant was diluted into water for analysis by UHPLC-UV. The chromatographic analysis was performed on a C18-based reversed-phase chromatography column at elevated temperature under gradient conditions, utilizing trifluoroacetic acid in water and acetonitrile. SGs were detected utilizing a UV detector set to 210 nm. A linear calibration curve was applied using a high-purity (>99%) Reb M standard as a reference solution.

Tables 9 and 10 show the percent recovery data from Reb M (>99%) study in 5% phosphoric acid matrix (pH<<1) stored at 40° C., in the presence of Rosmarinic Acid and Cichoric Acid respectively.

TABLE 9

| Experiment | Time (Days) | 0 | 1 | 2 | 3 | 7 |
|---|---|---|---|---|---|---|
| 0.1% Reb M No | % Reb M | 98 | 56 | 33 | 20 | 6 |
| SCs | Recovery | | | | | |
| 0.1% Reb M | % Reb M | 97 | 59 | 57 | 24 | 7 |
| 0.1% SCs | Recovery | | | | | |
| 1% Reb M 1% | % Reb M | 98 | 73 | 54 | 41 | 16 |
| SCs | Recovery | | | | | |
| 5% Reb M 5% | % Reb M | 97 | 81 | 66 | 55 | 27 |
| SCs | Recovery | | | | | |

TABLE 10

| Experiment | Time (Days) | 0 | 1 | 2 | 3 | 7 |
|---|---|---|---|---|---|---|
| 0.1% Reb M No | % Reb M | 98 | 56 | 33 | 20 | 6 |
| SCs | Recovery | | | | | |
| 0.1% Reb M | % Reb M | 97 | 59 | 36 | 23 | 6 |
| 0.1% SCs | Recovery | | | | | |
| 1% Reb M 1% | % Reb M | 98 | 72 | 54 | 40 | 13 |
| SCs | Recovery | | | | | |
| 5% Reb M 5% | % Reb M | 97 | 79 | 63 | 13 | 23 |
| SCs | Recovery | | | | | |

### Example 6

The steviol glycoside stabilizing compounds are themselves subject to degradation over time. The SCs can hydrolyze under acidic conditions to form caffeic acid. The SCs can also oxidize over time when exposed to oxygen. To study acidic degradation, the SCs were also quantified in the same experiment as outlined in Table 11. The SCs are much more resistant to acidic hydrolysis than the SGs, but the stability enhancement follows the same trend. At higher concentrations, the SGs and SCs both are more efficiently stabilized.

TABLE 11

| Experiment | Time (Days) | 0 | 1 | 2 | 3 | 7 |
|---|---|---|---|---|---|---|
| 0.1% Reb M No | % SC | — | — | — | — | — |
| SCs | Recovery | | | | | |
| 0.1% Reb M | % SC | 100% | 99% | 98% | 98% | 95% |
| 0.1% SCs | Recovery | | | | | |
| 1% Reb M 1% | % SC | 100% | 99% | 99% | 99% | 97% |
| SCs | Recovery | | | | | |
| 5% Reb M 5% | % SC | 100% | 100% | 99% | 99% | 99% |
| SCs | Recovery | | | | | |

### Example 7

To study the oxidative stability of the SEs, a solution of 500 ppm SC was prepared in pH 7 buffer. A second solution of 500 ppm SC with 500 ppm RM80 was prepared in pH 7 buffer. Both of these solutions were stirred aggressively and exposed to the oxygen in the atmosphere for 72 days. The results are summarized in Table 12 below and demonstrate that the presence of SGs will slow the oxidative degradation of the SCs.

TABLE 12

| Experiment | Time (Days) | 0 | 72 |
|---|---|---|---|
| 0.05% SCs | % SC Recovery | 100% | 17% |
| 0.05% SCs | % SC Recovery | 100% | 48% |
| 0.05% RM80 | | | |

### Example 8

It has been hypothesized that steviol glycosides (SGs) and steviol glycoside stabilizing compounds (SCs) will form a tight-binding complex in solution. If this is true, the magnetic environment of the complex would be substantially different than of the individual compounds dissolved in water. This would result in substantial shifting (Δδ>0.02 ppm) in their respective ¹H NMR spectra.

A total of four samples were prepared for this study, and they are listed below. Each sample was dissolved fully in water, with or without heat as noted below, flash frozen at −80° C., and then placed on a lyophilizer until dry. The dry powders were subsequently dissolved in D₂O at room temperature and analyzed by ¹H NMR and ¹³C NMR. The concentration of Sample 1 is substantially lower than Samples 2-4, as Reb M solubility in D₂O is much lower when SCs are not present, and this resulted in relatively poor quality spectra.
Sample 1: 10 mg Reb M in 1 mL water—heated in H₂O
Sample 2: 10 mg SE in 1 mL water—heated in H₂O
Sample 3: 10 mg Reb M+10 mg SE in 1 mL water—heated in H₂O
Sample 4: 10 mg Reb M+10 mg SE in 3 mL water—not heated in H₂O

Using the following numbering convention for SC molecules:

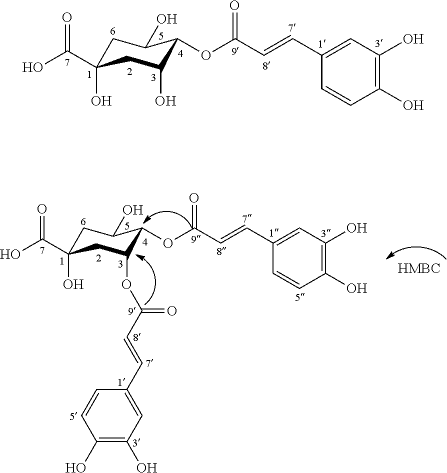

in this case, monocaffeoylquinic acid and dicaffeoylquinic acid. The observed signals are the sum of the mixture of isomers. The ¹H NMR data are shown in Tables 13 (showing ¹H NMR data with significant shifting in the caffeic acid moieties of the SCs) and 14 (Showing ¹H NMR data with significant shifting in the steviol core of the SG).

TABLE 13

| | | Average | Average |
|---|---|---|---|
| | | Shifting | Shifting |
| Protons | δ ppm range | Sample 3 vs. 2 | Sample 4 vs. 2 |
| C7′ and C7″ | 7.5-7.7 | +0.034 and | +0.029 and |
| | | +0.053 | +0.045 |
| C2′ and C2″ | 7.15-7.2 | +0.039 | +0.034 |
| C6′ and C6″ | 7.05-7.15 | +0.030 | +0.024 |
| C5′ and C5″ | 6.9-7.0 | +0.022 | +0.018 |
| C8′ and C8″ | 6.3-6.5 | +0.033 and | +0.028 and |
| | | +0.055 | +0.047 |

TABLE 14

| | | Average | Average |
|---|---|---|---|
| | | Shifting | Shifting |
| Protons | δ ppm range | Sample 3 vs 1 | Sample 4 vs 1 |
| C20 Methyl | 0.90 | −0.13 | −0.15 |
| C18 Methyl | 1.27 | −0.09 | −0.10 |

There is a substantial amount of shifting in both the SC signals and the SG signals when the mixture of molecules is present. This shifting is similar when the compounds are heated in water together versus when they are mixed at room temperature, but slightly greater when heated. The moieties which show the strongest shifting are the caffeic acid moieties of the SCs and the steviol backbone of the SGs, suggesting a strong interaction between the most hydrophobic regions of each molecule, leaving the glucose and quinic acid moieties free to interact with water, thus possibly increasing the stability of SGs and SCs.

The present invention provides for the following embodiments, the numbering of which is not to be construed as designating levels of importance:

Embodiment 1 relates to a composition comprising:
a steviol glycoside; and
a steviol glycoside stabilizing compound in an amount effective to reduce degradation of the steviol glycoside;
wherein the steviol glycoside stabilizing compound is at least one compound, and
isomers thereof, selected from the group consisting of:
   caffeic acid, an ester of caffeic acid, an ester of caffeic acid and quinic acid, an ester of caffeic acid and quinic acid comprising a single caffeic acid moiety (e.g., chlorogenic, cryptochlorogenic, and neochlorogenic acid; structures of each are provided herein), an ester of caffeic acid and quinic acid comprising more than one caffeic acid moiety (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid; structures of each are provided herein);
   ferulic acid, an ester of ferulic acid, an ester of ferulic acid and quinic acid, an ester of ferulic acid and quinic acid comprising a single ferulic acid moiety, an ester of ferulic acid and quinic acid comprising more than one ferulic acid moiety;
   3-(3,4-dihydroxyphenyl)lactic acid, a 3-(3,4-dihydroxyphenyl)lactic acid derivative, an ester of 3-(3,4-dihydroxyphenyl)lactic acid, an ester of a 3-(3,4-dihydroxyphenyl)lactic acid derivative,
   quinic acid, a quinic acid derivative, an ester of quinic acid, an ester of a quinic acid derivative;
   p-coumaric acid, an ester of p-coumaric acid, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety;
   sinapic acid, an ester of sinapic acid, an ester of sinapic acid and quinic acid, an ester of sinapic acid and quinic acid comprising a single sinapic acid moiety, an ester of sinapic acid and quinic acid comprising more than one sinapic acid moiety;
   tartaric acid, a tartaric acid derivative, an ester of tartaric acid, an ester of a tartaric acid derivative, and
   3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid.

Embodiment 2 relates to the composition of Embodiment 1, wherein the composition is an aqueous composition.

Embodiment 3 relates to the composition of Embodiments 1-2, wherein the amount of steviol glycoside stabilizing compound effective to reduce degradation of the steviol glycoside is an amount such that at least about 10 wt. % of an initial steviol glycoside remains when the stabilized steviol glycoside composition is subjected to storage for 7 days at 40° C. in 5% phosphoric acid.

Embodiment 4 relates to the composition of Embodiments 1-3, wherein the steviol glycoside comprises at least about 0.03 wt. % steviol glycoside.

Embodiment 5 relates to the composition of Embodiments 1-3, wherein the steviol glycoside comprises at least about 0.6 wt. % steviol glycoside.

Embodiment 6 relates to the composition of Embodiments 1-5, wherein the composition comprises a 1:0.3 to 1:3 ratio by weight of steviol glycoside to steviol glycoside stabilizing compound.

Embodiment 7 relates to the composition of Embodiments 1-6, wherein the composition has a pH of less than about 4.

Embodiment 8 relates to the composition of Embodiments 1-6, wherein the composition has a pH of less than about 1.

Embodiment 9 relates to the composition of Embodiments 1-8, wherein the composition is stored at room temperature.

Embodiment 10 relates to the composition of Embodiments 1-8, wherein the composition is stored at about 4° C.

Embodiment 11 relates to the composition of Embodiments 1-10, wherein the steviol glycoside is Rebaudioside A or Rebaudioside M.

Embodiment 12 relates to a beverage concentrate product comprising the composition of Embodiments 1-11, wherein the steviol glycoside comprises between about 1,800 ppm and about 10,000 ppm steviol glycoside.

Embodiment 13 relates to a liquid water enhancer product comprising the composition of Embodiments 1-11, wherein the steviol glycoside comprises between about 1.5 wt. % and about 3.5 wt. %. steviol glycoside.

Embodiment 14 relates to a liquid sweetener comprising the composition of Embodiments 1-11, wherein, the steviol glycoside comprises between about 1.0 wt. % and about 10 wt. % steviol glycoside.

1.-14. (canceled)

15. A method for stabilizing rebaudioside M in a steviol glycoside solution, the method comprising:
adding to an aqueous solution rebaudioside M; one or more dicaffeoylquinic acids or salts thereof; and one or more monocaffeoylquinic acids or salts thereof to form a steviol glycoside solution,
wherein rebaudioside M is at least 80% by weight of total steviol glycosides in the steviol glycoside solution;
wherein the steviol glycoside solution comprises a 1:0.3 to 1:3 ration by weight of steviol glycoside to the total of dicaffeoylquinic and monocaffeoylquinic acids and salts thereof; and
wherein at least 94% by weight of the initial rebaudioside M remains when the steviol glycoside solution is stored for 48 weeks at about 22° C. and pH 2.5.

16. The method of claim 15, wherein the steviol glycoside solution comprises at least 0.6% by weight steviol glycoside.

17. The method of claim 15, wherein the steviol glycoside solution had a pH less than 4.

18. The method of claim 15, wherein the steviol glycoside solution has a pH less than 1.

19. The method of claim 15, wherein in the steviol glycoside solution, the rebaudioside M, the monocaffeoylquinic acid or salt thereof, and the dicaffeoylquinic acid or salt thereof each have a shifted proton magnetic resonance signal of at least 0.02 relative to either compound alone.

20. The method of claim 15, wherein the monocaffeoylquinic is selected from the group consisting of chlorogenic acid, cryptochlorogenic acid, neochlorogenic acid, salts thereof, and combinations thereof.

21. The method of claim 15, wherein the dicaffeoylquinic is selected from the group consisting of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, salts thereof, and combinations thereof.

22. The method of claim 15, wherein the steviol glycoside solution additionally comprises one or more of 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, and salts thereof.

23. The method of claim 15, wherein the steviol glycoside solution comprises 1.5% by weight to 3.5% by weight steviol glycoside.
